(19) United States
(12) Patent Application Publication
(10) Pub. No.: US 2013/0225683 A1
(43) Pub. Date: Aug. 29, 2013

(54) Compounds and Pharmaceutical Compositions for Uses in Diabetes

(71) Applicants: Lyne Gagnon, Laval (CA); Brigette Grouix, Montreal (CA); Pierre Laurin, Ville Mont-Royal (CA)

(72) Inventors: Lyne Gagnon, Laval (CA); Brigette Grouix, Montreal (CA); Pierre Laurin, Ville Mont-Royal (CA)

(73) Assignee: PROMETIC BIOSCIENCES INC., Laval, Quebec (CA)

(21) Appl. No.: 13/882,373

(22) PCT Filed: Oct. 26, 2011
(86) PCT No.: PCT/CA2011/001180

§ 371 Date: Apr. 29, 2013

Related U.S. Application Data
(60) Provisional application No. 61/407,068, filed on Oct. 27, 2010.
(60) Provisional application No. 61/480,052, filed on Apr. 28, 2011.

(51) Int. Cl. A61K 31/192; A61K 31/155; A61K 31/426; A61K 31/20

(52) U.S. Cl. CPC A61K 31/192; A61K 31/20; A61K 31/155; A61K 31/426
USPC 514/559; 514/570; 514/568; 514/569; 514/571

(57) ABSTRACT

New uses for phenylketone carboxylate compounds and substituted aromatic compounds of Formula I, Formula I, IA, IB and IC, and their pharmaceutical acceptable salts are described for prevention or treatment of diabetes or a diabetes-related disorder in a subject in need thereof. Diabetes and diabetes-related disorder include Type I diabetes, Type II diabetes, maturity-onset diabetes of the young, latent autoimmune diabetes of adults (LADA), gestational diabetes, diabetic nephropathy, proteinuria, ketonuria, obesity, hyperglycemia, glucose intolerance, insulin resistance, hyperinsulinemia, hypercholesterolemia, hypertension, hyperlipoproteinemia, hyperlipidemia, hypertriglyceridemia, dyslipidemia, metabolic syndrome, syndrome X, diabetic neuropathy, diabetic retinopathy, hypoglycemia, cardiovascular disease, atherosclerosis, diabetic kidney disease, ketoacidosis, thrombotic disorders, sexual dysfunction, dermatopathy, edema, metabolic syndrome and renal disorders. The related pharmaceutical compositions and methods are also described. These compounds can be used in combination with comprising a therapeutic agent for lowering or controlling blood glucose level such as metformin or a thiazolidinedione.

### FIELD OF INVENTION

The present invention relates to the field of medicine. More particularly, the invention relates to methods, compositions and uses for prevention or treatment of diabetes or diabetes-related disorder such as Type I diabetes, Type II diabetes, maturity-onset diabetes of the young, latent autoimmune diabetes of adults (LADA), gestational diabetes, diabetic nephropathy, proteinuria, ketonuria, obesity, hyperglycemia, glucose intolerance, insulin resistance, hyperinsulinemia, hypercholesterolemia, hypertension, hyperlipoproteinemia, hyperlipidemia, hypertriglyceridemia, dyslipidemia, metabolic syndrome, syndrome X, diabetic neuropathy, diabetic retinopathy, hypoglycemia, cardiovascular disease, atherosclerosis, diabetic kidney disease, ketoacidosis, thrombotic disorders, sexual dysfunction, dermatopathy, edema, metabolic syndrome and renal disorders.

### BACKGROUND OF INVENTION

### Diabetes

Diabetes is caused by multiple factors and is characterized by elevated levels of plasma glucose (hyperglycemia) in the fasting state. There are two generally recognized forms of diabetes: Type I diabetes, or insulin dependent diabetes, in which patients produce little or no insulin and Type II diabetes, or noninsulin-dependent diabetes wherein patients produce insulin, while at the same time demonstrating hyperglycemia. Type I diabetes is typically treated with exogenous insulin administered via injection. However, Type II diabetics often present “insulin resistance”, such that the effect of insulin in stimulating glucose and lipid metabolism in the main insulin-sensitive tissues, namely muscle, liver and adipose tissues, is diminished and hyperglycemia results.

Persistent or uncontrolled hyperglycemia that occurs in diabetes is associated with increased morbidity and premature mortality. Abnormal glucose homeostasis is also associated, both directly and indirectly, with obesity, hypertension and alterations in lipid, lipoprotein and apolipoprotein metabolism. Type II diabetics are at increased risk of cardiovascular complications such as atherosclerosis, coronary heart disease, stroke, peripheral vascular disease, hypertension, nephropathy, retinopathy and also neuropathy. Many patients who have insulin resistance, but have not developed Type II diabetes, are also at risk of developing symptoms referred to as “Syndrome X”, or “Metabolic Syndrome”. Metabolic syndrome is characterized by insulin resistance, along with abdominal obesity, hyperinsulinemia, high blood pressure, low HDL (high density lipoproteins) and high VLDL (very low density lipoprotein), hypertriglyceridemia and hyperuricemia. Whether or not they develop overt diabetes, these patients are at increased risk of developing cardiovascular complications.

Current treatments for diabetes include: insulin, insulin secretagogues, such as sulphonylureas, which increase insulin production from pancreatic β-cells; glucose-lowering effectors, such as metformin which reduce glucose production from the liver; activators of the peroxisome proliferator-activated receptor-γ (PPAR-γ), such as the thiazolidinediones, which enhances insulin action; and a-glucuronidase inhibitors which interfere with gut glucose production. However, there are some deficiencies associated with these treatments. For example, sulphonylureas and insulin injections can be associated with hypoglycemia and weight gain. Responsiveness to sulphonylureas is often lost over time. Gastrointestinal problems are observed with metformin and a-glucosidase. Finally, PPAR-γ agonists may cause increase weight and edema.

### Diabetic Nephropathy

Diabetic nephropathy also known as Kimmelstiel-Wilson syndrome and intercapillary glomerulonephritis, is a progressive kidney disease caused by angiopathy of capillaries in the kidney glomeruli. It is characterized by nodular glomerulosclerosis due to longstanding diabetes mellitus and is a prime cause for dialysis in many Western countries. The syndrome can be seen in patients with chronic diabetes. The disease is progressive and may cause death two or three years after the initial lesions and is more frequent in women. Diabetic nephropathy is the most common cause of chronic kidney failure and end-stage kidney disease in the United States. People with both Type I and Type II diabetes are at risk. The risk is higher if blood-glucose levels are poorly controlled. However, once nephropathy develops, the greatest rate of progression is seen in patients with poor control of their blood pressure.

Diabetic nephropathy is clinically well defined and is characterized by proteinuria, hypertension, edema and renal insufficiency. There are limited treatment options for diabetic nephropathy. Current treatments are primarily directed to improving complications of the diseases as follows: 1) control of blood-pressure (ACE-inhibitors inhibitors or Angiotensin receptor blockers (ARBs)); 2) Control of glycemic values; and 3) “lipoprotein diet”, exercise or other life styles modifications. However, there is an important need for better drugs and treatments since current treatment may have limited impact on the progressive decline in kidney function and patients still progress to renal replacement therapy, either dialysis or renal transplantation.

Hyperlipidemia is a major complication of diabetic nephropathy and is a determinant of progression of renal disorder in diabetes. Hyperlipidemia is a pathogenic factor for diabetic nephropathy and clinical studies involving therapeutic interventions for hyperlipidemia suggest the importance of this approach in at least slowing the progression of diabetic renal disorder (Rosario and Prabhakar (2006), Current Diabetes Reports, 6:455-462). Therefore, there is a need for methods and compounds for modulating blood lipids levels, and more particularly reducing levels of harmful serum lipid levels, especially cholesterol and triglycerides in diabetic patients.

Various patent documents have disclosed compounds reportedly useful for diabetes and diabetic nephropathy. Examples include International PCT patent publications WO 2004/1076276, WO 2004/041266, WO2005/086661; WO 2010/127440; European patent publications EP 1 630 152 and EP 1 559 422; and U.S. Patent Publications No. 2004/0038126 and No. 2006/004012. U.S. Patent Publication No. 2007/0066647 discloses compounds useful for treating metabolic disorders.

The present invention addresses the needs for new treatment methods, compounds and pharmaceutical compositions for treating patients with diabetes, patients with diabetic nephropathy and patients with disorders and conditions associated with abnormal levels of glucose, insulin, ketone bodies, plasma lipoprotein and/or triglycerides.

Additional features of the invention will be apparent from a review of the disclosure, figures and description of the invention herein.

### BRIEF SUMMARY OF THE INVENTION

The present invention relates to methods, compounds and compositions for prevention and/or treatment of diabetes or a diabetes-related disorder in a subject in need thereof.

Particular aspects of the invention relates to the use of compounds according to any of Formula 1, 1A, 1B and 1C as defined herein, and pharmaceutically acceptable salts thereof. The salt may be sodium, potassium, calcium, magnesium or lithium. Preferably, the compound is the sodium salt of or Compound I or the sodium salt of Compound XIV. Specific examples of compounds according to the invention are represented in Table 1.)

One particular aspect of the invention concerns the use of a compound represented by any of the formulas 1, 1A, 1B and 1C as defined herein or a pharmaceutically acceptable salt thereof, for prevention or treatment of diabetes or a diabetes-related disorder in a subject in need thereof, or for the manufacture of a medicament for prevention or treatment of diabetes or a diabetes-related disorder. Another aspect of the invention concerns a pharmaceutical composition comprising a compound represented by any of the formulas 1, 1A, 1B and 1C as defined herein or a pharmaceutically acceptable salt thereof for prevention or treatment of diabetes or a diabetes-related disorder.

In an embodiment, the pharmaceutically acceptable salt is a base addition salt. The base addition salt comprises a metal counterion which is preferably sodium, potassium, magnesium, calcium or lithium.

In another particular aspect of the invention, the compound is any one of Compounds I-XXXIV as defined in Table 1. In embodiments of the invention, the compound is Compound I, II, V, VIII, XIV, XXIII or XXVI. In another embodiment, the compound is Compound I. In a further embodiment, the compound is Compound XIV.

Another aspect of the invention concerns the use of a compound represented by any of the formulas 1, 1A, 1B and 1C as defined herein or a pharmaceutically acceptable salt thereof, or a pharmaceutical composition comprising a compound represented by any of the formulas 1, 1A, 1B and 1C as defined herein or a pharmaceutically acceptable salt thereof, and a pharmaceutically acceptable carrier, for prevention or treatment of diabetes or diabetes-related disorder. Such diabetes or diabetes-related disorder is preferably a Type I diabetes, Type II diabetes, maturity-onset diabetes of the young, latent autoimmune diabetes of adults (LADA), gestational diabetes, diabetic nephropathy, proteinuria, ketonuria, obesity, hyperglycemia, glucose intolerance, insulin resistance, hyperinsulinemia, hypercholesterolemia, hypertension, hyperlipoproteinemia, hyperlipidemia, hypertriglyceridemia, dyslipidemia, metabolic syndrome, syndrome X, diabetic neuropathy, diabetic retinopathy, hypoglycemia, cardiovascular disease, atherosclerosis, diabetic kidney disease, ketoacidosis, thrombotic disorders, sexual dysfunction, dermatopathy, edema, metabolic syndrome or renal disorders. In a preferred embodiment, the use is for prevention or treatment of Type II diabetes. In another preferred embodiment, the use if for prevention or treatment of diabetic nephropathy. In a further preferred embodiment, the use if for prevention or treatment is proteinuria. In another further preferred embodiment, the use if for prevention or treatment of ketonuria. In a further aspect of the invention, the use is also for decreasing ketone bodies in the urine of a subject in need thereof. In another aspect of the invention, the use is also for increasing glomerular filtration rate (GFR) in a subject in need thereof. In another aspect of the invention, the use is also for increasing insulin secretion or increasing insulin sensitivity in a subject in need thereof. In still a further aspect of the invention, the use is also for decreasing insulin resistance in a subject in need thereof. In yet another aspect of the invention, the use is also for decreasing hyperglycemia in a subject in need thereof.

Another related aspect of the invention, the compound represented by any of the formulas 1, 1A, 1B and 1C as defined herein or a pharmaceutically acceptable salt thereof, is used in combination with a therapeutic agent for lowering or controlling blood glucose level, which is preferably metformin or thiazolidinedione. In a preferred embodiment, the pharmaceutical composition is adapted for oral administration.

Another related aspect of the invention concerns a method for prevention or treatment of diabetes or a diabetes-related disorder in a subject in need thereof, said method comprising administering a compound represented by any of the formulas 1, 1A, 1B and 1C as defined herein or a pharmaceutically acceptable salt thereof, or a pharmaceutical as defined herein.

Additional aspects of the invention concerns the use for positively affecting in a human subject in need thereof at least one pancreatic function parameter, wherein said at least one pancreatic function parameter is selected from the group consisting of: (i) size, growth and/or secretion activity of islets of Langerhans; (ii) size, growth and/or secretion activity of beta-cells; (iii) insulin secretion; (iv) insulin blood levels and (v) glucose blood levels; said use comprising administering to a human subject a compound as defined in any one of claims 1-7, or a pharmaceutically acceptable salt, thereof; whereby said administration positively affects in said human subject at least one pancreatic function parameter.

Additional aspects of the invention concerns the use for (1) restoration of beta-cell mass and/or function; (2) prevention and/or treatment of type I diabetes; (3) prevention and/or treatment of latent autoimmune diabetes; (4) preservation and/or increase in the number of functional insulin-producing cells; and/or (5) decrease of resistance to insulin and/or increase to insulin sensitivity.

Additional aspects of the invention concerns the use is also for decreasing the rate of the increase of proteinuria; (ii) decreasing the rate of the rise in serum creatinine; and (iii) decreasing the fall of creatinine clearance or GFR.

Additional aspects of the invention concerns the use for (i) decreasing the rate of the increase of proteinuria; (ii) decreasing the rate of the rise in serum creatinine; and (iii) decreasing the fall of creatinine clearance or GFR.

Further aspects of the invention will be apparent to a person skilled in the art from the following description, claims, and generalizations herein.

### BRIEF DESCRIPTION OF THE FIGURES

FIG. 1 is a bar graph showing the effect of Compound I on blood glucose concentration in Streptozotocin-induced diabetes.

FIG. 2 is a bar graph showing the effect of Compound I on urine ketone bodies in Streptozotocin-induced diabetes.

FIG. 3 is a bar graph showing the effect of Compound I on urine proteins concentration in Streptozotocin-induced diabetes.

FIG. 4 is a bar graph showing the effect of Compound I on kidney improvement as demonstrated by increase in GFR in Streptozotocin-induced diabetes.

FIG. 5 is a bar graph showing the effect of Compound XIV on blood glucose concentration in 5/6 nephrectomized rats.

FIG. 6 is a bar graph showing the effect of Compound XIV on urine proteins concentration in 5/6 nephrectomized rats.

FIG. 7 is a line graph showing the effect of Compound I on percentage of increase of serum glucose concentration in diabetic db/db mice.

FIG. 8 is a line graph showing the effect of Compound I on blood glucose concentration in 5/6 nephrectomized rats.

FIG. 9 is a bar graph showing the effect of Compound I on kidney improvement as demonstrated by increase in GFR in diabetic db/db mice.

### DETAILED DESCRIPTION OF THE INVENTION

The present discloses pharmaceutical applications of compounds of Formula 1, 1A, 1B and 1C and compositions comprising same. Some compounds according to the invention may be broadly classified as substituted phenyl (phenoxy, thiophenoxy, anilino) benzoic, acetic or propionic acids.

### A) Compounds of the Invention

### Compounds of the Invention:

According to one aspect, the invention concerns the pharmaceutical uses in the prevention and/or treatment of diabetes or diabetes-related disorder of compounds represented by Formula I, or pharmaceutically acceptable salts thereof:

Cy-Q Formula I

wherein

### Cy is

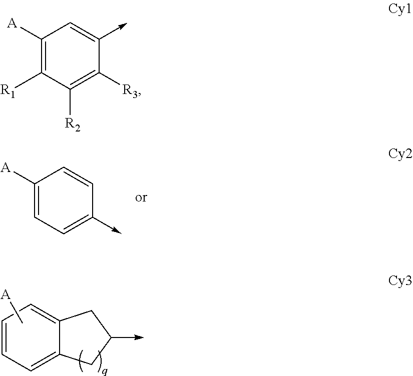

where
q is 1, 2 or 3
→ represents a covalent bond connecting Cy to Q;
A is
   1) C₁-C₆ alkyl,
   2) C₂-C₆ alkenyl,
   3) C₁-C₇ alkyl-Y—,
   4) C₁-C₇ alkyl-OC(O)—, or
   5) C₁-C₇ alkyl-CH(OH)—,
      where Y is O, S or C(O);
R₁, R₂ and R₃ are independently selected from H, F, Cl or OH;
when Cy is Cy1 or Cy2, then Q is
   1) C(O)OH,
   2) C(CH₃)₂C(O)OH,
   3) (CH₂)mC(O)OH,
   4) ZCH(C(O)OH)C₁-C₈ alkyl,
   5) Z(CH₂)mC(O)OH, or
   6) CH₂CH(C(O)OH)C₁-C₈ alkyl,
   where
      Z is 0 or S,
      m is 1 or 2; and
when Cy is Cy3, then Q is C(O)OH.

According to another aspect, the invention concerns the pharmaceutical uses in the prevention and/or treatment of diabetes or diabetes-related disorder of compounds represented by Formula 1A, or pharmaceutically acceptable salts thereof:

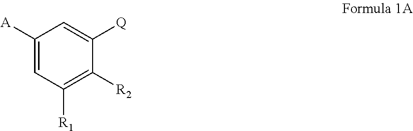

wherein
A is
   1) C₁-C₆ alkyl, or
   2) C₁-C₆ alkenyl;
R₁ and R₂ are independently selected from H, F, Cl or OH;
Q is
   1) C(O)OH,
   2) C(CH₃)₂C(O)OH, or
   3) (CH₂)mC(O)OH,
   where is 1 or 2.

According to another aspect, the invention concerns the pharmaceutical uses in the prevention and/or treatment of diabetes or diabetes-related disorder of compounds represented by Formula 1B, or pharmaceutically acceptable salts thereof;

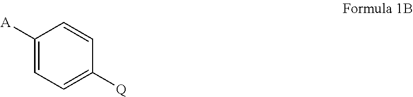

wherein
A is
   1) C₆ alkyl, or
   2) C₁-C₆ alkenyl;
Q is
   1) C(O)OH, or
   2) (CH₂)mC(O)OH,
   where m is 1 or 2.

According to another aspect, the invention concerns the pharmaceutical uses in the prevention and/or treatment of diabetes or diabetes-related disorder of compounds represented by Formula 1C or pharmaceutically acceptable salts thereof;

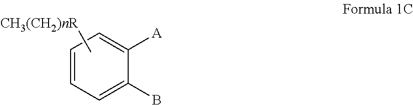

wherein
n is 2, 3, 4, 5 or 6;
R is —C(O)—, —OC(O)—, —CH(OH)—, O;
A is (CH₂)mC(O)OH, W(CH₂)mC(O)OH or YCH(C(O)OH)(CH₂)ₚCH₃ when B is H;
B is (CH₂)mC(O)OH, W(CH₂)mC(O)OH or YCH(C(O)OH)(CH₂)ₚCH₃ when A is H; or A and B are covalently bonded to form a 5-, 6- or 7-membered cycloalkyl substituted with a C(O)OH group;
where
   W is O or S,
   Y is O, S or CH₂,
   m is 0, 1 or 2, and
   p is 1, 2, 3, 4, 5, 6 or 7.

As used herein, the term “alkyl” is intended to include both branched and straight chain saturated aliphatic hydrocarbon groups having the specified number of carbon atoms, for example, C₁-C₈ as in C₁-C₈ alkyl is defined as including groups having 1, 2, 3, 4, 5, 6, 7 or 8; C₁-C₇ as in C₁-C₇ alkyl is defined as including groups having 1, 2, 3, 4, 5, 6, or 7; C₁-C₆ as in C₁-C₆ alkyl is defined as including groups having 1, 2, 3, 4, 5, or 6, carbons in a linear or branched arrangement; for example, C₁-C₄ as in C₁-C₄ alkyl is defined as including groups having 1, 2, 3, or 4 carbon atoms in a linear or branched arrangement; or C₁-C₃ as in C₁-C₃ alkyl is defined as including groups having 1, 2, or 3. Examples of alkyl defined above include, but are not limited to, methyl, ethyl, n-propyl, i-propyl, n-butyl, t-butyl, butyl, pentyl, hexyl, heptyl and octyl.

As used herein, the term, “alkenyl” is intended to mean unsaturated straight or branched chain hydrocarbon groups having the specified number of carbon atoms therein, and in which at least two of the carbon atoms are bonded to each other by a double bond, and having either E or Z regiochemistry and combinations thereof. For example, C₂-C₆ as in C₂-C₆ alkenyl is defined as including groups having 2, 3, 4, 5, or 6 carbons in a linear or branched arrangement, at least two of the carbon atoms being bonded together by a double bond, or C₂-C₄ as in C₂-C₄ alkenyl is defined as including groups having 2, 3, or 4 carbons in a linear or branched arrangement, at least two of the carbon atoms being bonded together by a double bond. Examples of alkenyl include ethenyl (vinyl), 1-propenyl, 2-propenyl, and 1-butenyl.

As used herein, the term “cycloalkyl” is intended to mean a monocyclic saturated aliphatic hydrocarbon group having the specified number of carbon atoms therein, for example, as in C₅-C₇ cycloalkyl is defined as including groups having 5, 6 or 7 carbons in a monocyclic arrangement. Examples of C₅-C₇ cycloalkyl include, but are not limited to, cyclopentyl, cyclohexyl and cycloheptyl.

Examples of compounds of Formula I include, but are not limited to, the compounds listed in Table 1 hereinafter.

TABLE 1
Examples of compounds of Formula 1

| Compound | Structure |
|---|---|
| I | 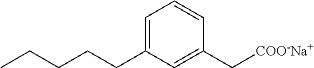 |
| II | 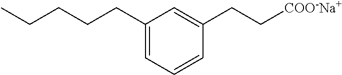 |
| III | 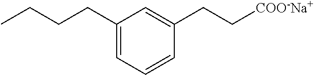 |
| IV | 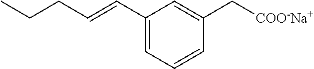 |
| V | 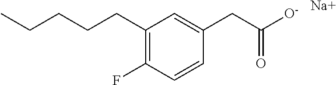 [SMILES: [Na+] ; CCCCCc1cc(CC(=O)[O-])ccc1F] |
| VI | 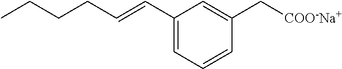 |
| VII | 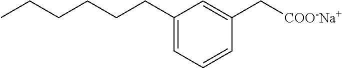 |
| VIII | 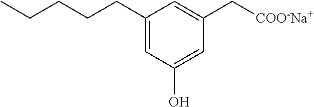 |
| IX | 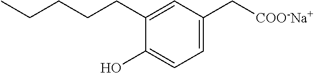 |
| X | 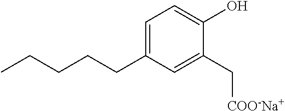 |
| XI | 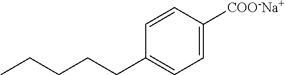 |
| XII | 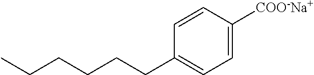 |
| XIII | 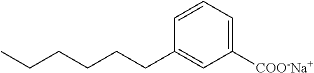 |
| XIV | 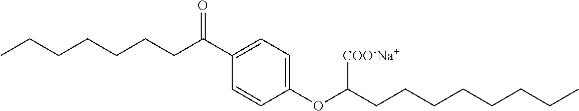 |
| XV | 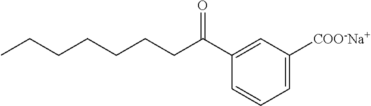 |
| XVI | 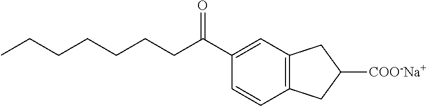 |
| XVII | 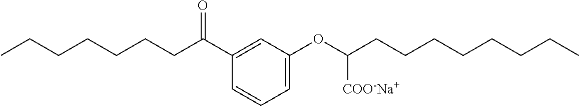 |
| XVIII | 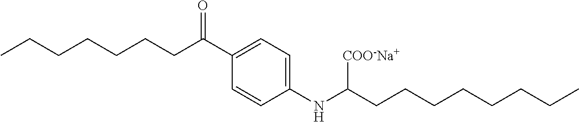 |
| XIX | 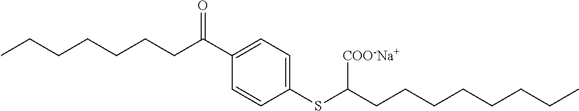 |
| XX | 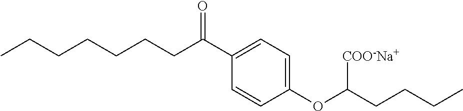 |
| XXI | 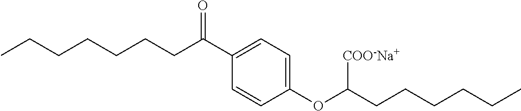 |
| XXII | 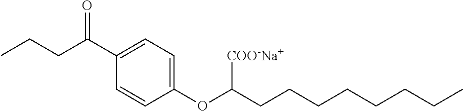 |
| XXIII | 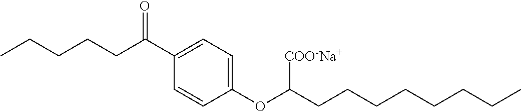 |
| XXIV | 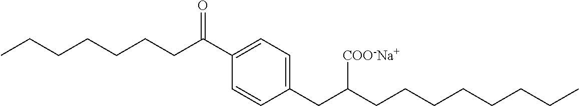 |
| XXV | 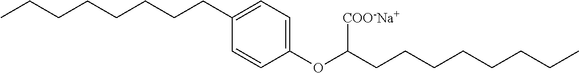 |
| XXVI | 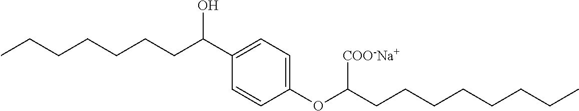 |
| XXVII | 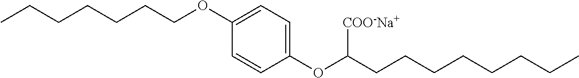 |
| XXVIII | 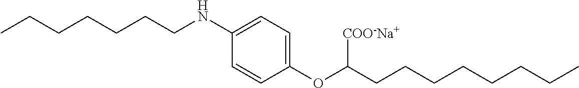 |
| XXIX | 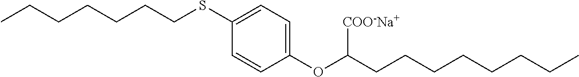 |
| XXX | 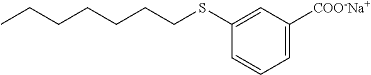 |
| XXXI | 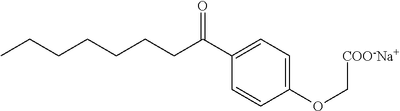 |
| XXXII | 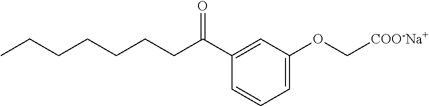 |
| XXXIII | 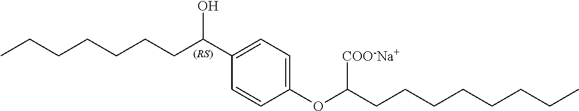 |
| XXXIV | 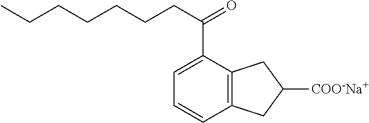 |

The Applicants have described elsewhere compounds whose structure is related to the structure of some of the compounds of the present invention. Reference is made for instance to the compounds disclosed in Table 2 of international PCT application No. PCT/CA2010/000677 filed on May 3, 2010 (published as WO 2010/127440) entitled “Substituted aromatic compounds and pharmaceutical uses thereof” which is incorporated herein by reference in its entirety. Accordingly, in particular embodiments any one or all the Compounds I to XV and XVIII disclosed in Table 2 of WO 2010/127440 are excluded from the scope of the present invention. Similarly, in particular embodiments, the uses of compounds of Formula 1, 1A, 1B and 10 as described herein in medical applications encompassing the prevention or treatment of an “oxidative stress related disorder” as defined WO 2010/127440 are excluded from the scope of the present invention. In addition, in particular embodiments, diabetic subjects suffering from diabetes caused by oxidative stress (i.e. subjects suffering from an imbalance between the production of reactive oxygen species and ability to readily detoxify the reactive intermediates or easily repair the resulting damages) are excluded from the scope of the present invention.

In particular embodiments, the uses of compounds of Formula 1, 1A, 1B and 1C as described herein in medical applications encompassing the prevention or treatment of nephropathies and renal disorders (e.g., diabetic nephropathy) caused by fibrosis are excluded from the scope of the present invention

### Salts

As used herein, the term “pharmaceutically acceptable salt” is intended to mean base addition salts. Example of pharmaceutically acceptable salts are also described, for example, in Berge et al., “Pharmaceutical Salts”, J. Pharm. Sci. 66, 1-19 (1977). Pharmaceutically acceptable salts may be synthesized from the parent agent that contains an acidic moiety, by conventional chemical methods. Generally, such salts are prepared by reacting the free acid forms of these agents with a stoichiometric amount of the appropriate base in water or in an organic solvent, or in a mixture of the two. Salts may be prepared in situ, during the final isolation or purification of the agent or by separately reacting a purified compound of the invention in its free acid form with the desired corresponding base, and isolating the salt thus formed.

The pharmaceutically acceptable salt of the compounds of Formula 1, 1A, 1B and 1C may be selected from the group consisting of base addition salts of sodium, potassium, calcium, magnesium and lithium. In preferred embodiment, the base addition salt is sodium. Is some embodiments, the compounds are the sodium salts listed in Table 1 hereinbefore. Preferably the compound is selected from Compounds I and XIV as defined herein. More preferably, the compound is Compound I as defined herein.

All acid, salt and other ionic and non-ionic forms of the compounds described are included as compounds of the invention. For example, if a compound is shown as an acid herein, the salt forms of the compound are also included. Likewise, if a compound is shown as a salt and the acid forms are also included.

### Prodrugs

In certain embodiments, the compounds of the present invention as represented by generalized Formula 1, 1A, 1B and 1C wherein said compounds are present in the free carboxylic acid form, may also include all pharmaceutically acceptable salts, isosteric equivalents such as tetrazole and prodrug forms thereof. Examples of the latter include the pharmaceutically acceptable esters or amides obtained upon reaction of alcohols or amines, including amino acids, with the free acids defined by Formula 1, 1A, 1B and 1C.

### Chirality

The compounds of the present invention, their pharmaceutically acceptable salts, or prodrugs thereof, may contain one or more asymmetric centers, chiral axes and chiral planes and may thus give rise to enantiomers, diastereomers, and other stereoisomeric forms and may be defined in terms of absolute stereochemistry, such as ®- or (S)- or, as (D)- or (L)- for amino acids. The present invention is intended to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. The racemic mixtures may be prepared and thereafter separated into individual optical isomers or these optical isomers may be prepared by chiral synthesis. The enantiomers may be resolved by methods known to those skilled in the art, for example by formation of diastereoisomeric salts which may then be separated by crystallization, gas-liquid or liquid chromatography, selective reaction of one enantiomer with an enantiomer specific reagent. It will also be appreciated by those skilled in the art that where the desired enantiomer is converted into another chemical entity by a separation technique, an additional step is then required to form the desired enantiomeric form. Alternatively specific enantiomers may be synthesized by asymmetric synthesis using optically active reagents, substrates, catalysts, or solvents or by converting one enantiomer to another by asymmetric transformation.

Certain compounds of the present invention may exist in Zwitterionic form and the present invention includes Zwitterionic forms of these compounds and mixtures thereof.

### Hydrates

In addition, the compounds of the invention also may exist in hydrated and anhydrous forms. Hydrates of any of the formulas described herein are included as compounds of the invention which may exist as a monohydrate or in the form of a polyhydrate.

### B) Methods of Preparation

In general, all compounds of the present invention may be prepared by any conventional methods, using readily available and/or conventionally preparable starting materials, reagents and conventional synthesis procedures. Of particular interest is the work of Hundertmark, T.; Littke, A. F.; Buchwald, S. L.; Fu, G. C. Org. Lett. 2000, 12, pp. 1729-1731.

The exemplification section hereinafter provides general schemes and specific, but non limitative, examples for the synthesis of Formula 1, 1A, 1B and 1C. Those skilled in the art may also refer to the Applicants published PCT application WO 2010/127440 (incorporated herein by reference in its entirety) disclosing compounds whose structure is related to the structure of some of the compounds of the present invention.

### C) Pharmaceutical Applications

As indicated and exemplified herein, the compounds of the present invention have beneficial pharmaceutical properties and these compounds may have useful pharmaceutical applications in the prevention and/or treatment of various diseases and/or conditions in a subject. Medical and pharmaceutical applications contemplated by the inventors include, but are not limited to, those diseases and conditions where abnormal blood levels of glucose, abnormal blood levels of insulin, abnormal levels of ketone bodies in the urine, abnormal levels of plasma lipoprotein and/or abnormal blood levels of triglycerides are an issue. Examples of such diseases and conditions include, but are not limited to, Type I diabetes, Type II diabetes, maturity-onset diabetes of the young, latent autoimmune diabetes of adults (LADA), gestational diabetes, ketonuria, diabetic nephropathy and other renal disorders, obesity, hyperglycemia, glucose intolerance, insulin resistance, hyperinsulinemia, hypercholesterolemia, hypertension, hyperlipoproteinemia, hyperlipidemia, hypertriglyceridemia, dyslipidemia, metabolic syndrome, syndrome X, diabetic neuropathy, diabetic retinopathy, hypoglycemia, cardiovascular disease, atherosclerosis, kidney disease, ketoacidosis, thrombotic disorders, nephropathy, sexual dysfunction, dermatopathy, dyspepsia, cancer and edema.

The term “subject” includes living organisms in which abnormal blood levels of glucose, abnormal blood levels of insulin, abnormal levels of ketone bodies in the urine, abnormal levels of plasma lipoprotein and/or abnormal blood levels of triglycerides can occur, or which are susceptible to such conditions. The term “subject” includes animals such as mammals or birds. Preferably, the subject is a mammal. More preferably, the subject is a human. Most preferably, the subject is a human patient in need of treatment, including but not limited to a diabetic patient.

As used herein, “preventing” or “prevention” is intended to refer to at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease). Biological and physiological parameters for identifying such patients are provided herein and are also well known by physicians.

The terms “treatment of” or “treating a subject” includes the application or administration of a compound of the invention to a subject (or application or administration of a compound of the invention to a cell or tissue from a subject) with the purpose of delaying, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being. In some embodiments, the term “treating” can include increasing a subject's life expectancy and/or delay before additional treatments are required (e.g., surgery, dialysis or transplantation).

### Diabetes

Addressing “diabetes-related disorders” is among the medical and pharmaceutical applications contemplated by the present invention. Diabetes mellitus, often simply referred to as diabetes, is characterized by a group of metabolic diseases in which a person has high blood sugar, either because the body does not produce enough insulin, or because cells do not respond to the insulin that is produced. As used herein, the term “diabetes-related disorders” refers to any disorder and condition associated with abnormal blood levels of glucose, abnormal blood levels of insulin, abnormal levels of ketone bodies in the urine, abnormal levels of plasma lipoprotein and/or abnormal blood levels of triglycerides can occur, including but not limited to, type II diabetes, ketonuria, obesity, hyperglycemia, glucose intolerance, insulin resistance, hyperinsulinemia, hypercholesterolemia, hypertension, hyperlipoproteinemia, hyperlipidemia, hypertriglyceridemia, dyslipidemia, metabolic syndrome, syndrome X, diabetic neuropathy, diabetic retinopathy, hypoglycemia. In some embodiments, it may also include diseases and conditions derived from diabetes existence such as cardiovascular disease, atherosclerosis, kidney disease, ketoacidosis, thrombotic disorders, nephropathy, sexual dysfunction, dermatopathy and edema. In preferred embodiments, the present invention relates to methods, compounds and compositions where high blood sugar is a medical problem, e.g. type II diabetes.

### Pancreatic Diseases

One additional aspect of the invention concerns the use of a compound represented by any of the formulas as defined herein for use in preventing and/or treating a pancreatic or beta-cell related disease. The term “pancreatic disease” or “beta-cell related disease” means any alteration in normal physiology and/or function of the pancreas. As used herein, it more particularly refers to the endocrine function of the pancreas which relates to the production and/or secretion of insulin and maintenance of appropriate blood glucose levels. These terms also encompass all clinical-pathological conditions or diseases that are directly or indirectly related to an undesirably high glycemia or undesirably low levels of blood insulin. This can result from a wide range of acute and chronic conditions and events, including physical, chemical or biological injury, insult, trauma or disease, such as for example type I diabetes, type II diabetes, maturity-onset diabetes of the young, latent autoimmune diabetes of adults (LADA), gestational diabetes, obesity, hypertension, metabolic syndrome, renal disorders, etc. The term pancreatic disease” or “beta-cell related disease” also include but are not limited to diseases and conditions where preventing loss or stimulating neogenesis of islets of Langerhans and/or beta-cells, stabilizing the insulin secreting function of the pancreas would be desirable (e.g., type I and type II diabetes). In preferred embodiment, the pancreatic disease or beta-cell related disease is Type II diabetes.

As used herein, the term “pancreas” refers to the large, elongated, racemose gland situated transversely behind the stomach, between the spleen and the duodenum. The pancreas is composed of an endocrine portion (the pars endocrina) and an exocrine portion (the pars exocrina). The pars endocrina, which contains the islets of Langerhans, produces and secretes proteins, including insulin, directly into the blood stream. The pars exocrina contains secretory units and produces and secretes a pancreatic juice, which contains enzymes essential to protein digestion, into the duodenum.

By “islet cell” is meant a cell having a phenotype similar to the hormone-producing cells normally comprising the pancreatic islets of Langerhans, and generally characterized by the expression of markers that normally distinguishing the cells in the pancreatic islets of Langerhans from other pancreatic cells, such as insulin, glucagon, somatostatin, pancreatic polypeptide, or islet amyloid polypeptide (IAPP or amylin).

By “beta-cell”, or “β-cell” is meant a pancreatic islet cell having a phenotype characterized by the expression of markers that normally distinguish the beta-cells from the other pancreatic islets cells, such as insulin, Nk×6.1 or glucokinase.

Without being bound by any theory, the compounds of the invention may increase the regeneration or prevent the apoptosis of islets of Langerhans to prevent or ameliorate the symptoms of diabetes mellitus. The compounds and compositions of the invention may also: (1) restore beta-cell mass and function in an individual in need thereof; (2) prevent or treat type I diabetes in an individual in need thereof; (3) prevent or treat latent autoimmune diabetes of adults (LADA) in an individual in need thereof; (4) treat type II diabetes by preserving or increasing the number of functional insulin-producing cells (e.g., beta-cells) and/or (5) decrease resistance to insulin and/or increasing insulin sensitivity. The present invention encompasses these and other possible mechanisms of action.

A related aspect of the invention concerns methods for positively affecting in a human subject in need thereof at least one pancreatic function parameter, such as: (i) size, growth and/or secreting activity of islets of Langerhans; (ii) size, growth and/or secreting activity of beta-cells; (iii) insulin secretion; (iv) insulin blood levels and (v) glucose blood levels. The method comprises administering to said human subject a compound represented by any of Formula 1, 1A, 1B and 1C described herein or a pharmaceutically acceptable salt, whereby the administration positively affects in the human subject at least one of said pancreatic function parameter.

Preferably, the administration of one or more compounds according to the invention provides in the human subject at least one of the following benefits: (1) restoration of beta-cell mass and/or function; (2) prevention and/or treatment of type I diabetes; (3) prevention and/or treatment of latent autoimmune diabetes; (4) preservation and/or increase in the number of functional insulin-producing cells; and/or (5) decrease of resistance to insulin and/or increase to insulin sensitivity.

Accordingly, related aspects of the invention concerns the uses of compounds as defined herein for modulating glucose, insulin and/or lipid levels in a subject, and more particularly in subjects suffering from obesity; hypoglycemia, hyperglycemia, and/or glucose intolerance; insulin resistance and/or hyperinsulinemia; and dyslipidemia (e.g., hyperlipidemia, hypercholesterolemia, hyperlipoproteinemia, and/or hypertriglyceridemia).

### Diabetic Nephropathy

Diabetes mellitus can produce nephropathies and particular aspects of the invention concerns the use of a compound represented by any of the formulas as defined herein for use in preventing and/or treating nephropathies associated with diabetes. The terms “nephropathy” or “nephropathies” encompass all clinical-pathological changes in the kidney which may be caused directly or indirectly by diabetes, including but not limited to kidney fibrosis, glomerular diseases (e.g., glomerulosclerosis, glomerulonephritis), chronic renal insufficiency, end stage renal disease and/or renal failure.

Some aspects of the present invention relate to methods, compounds and compositions as defined herein and their uses for the prevention and/or treatment of diabetic nephropathy, hypertensive nephropathy, and other types of nephropathies such as analgesic nephropathy, immune-mediated glomerulopathies (e.g., IgA nephropathy or Berger's disease, lupus nephritis), ischemic nephropathy, HIV-associated nephropathy, membranous nephropathy, glomerulonephritis, glomerulosclerosis, radiocontrast media-induced nephropathy, toxic nephropathy, analgesic-induced nephrotoxicity, cisplatin nephropathy, transplant nephropathy, and other forms of glomerular abnormality or injury; glomerular capillary injury (tubular fibrosis). In some embodiments, the terms “nephropathy” or “nephropathies” refers specifically to a disorder or disease where there is either the presence of proteins (i.e., proteinuria) in the urine of a subject and/or the presence of renal insufficiency.

According to some embodiments, the present invention concerns methods, compounds and compositions for preventing or treating diabetic nephropathy in a subject in need thereof. Diabetic nephropathy is a clinically well-defined pathology characterized by proteinuria, hypertension, edema and renal insufficiency. Characteristic aspects of diabetic nephropathy include glomerulosclerosis, modification of the vascular structure, and tubulointerstitial disease. The first clinical evidence of diabetic nephropathy is often the presence of albuminuria in the urine, e.g., microalbuminuria or macroalbuminuria.

As is known, diabetic nephropathy is typically characterized by the following: 1) glomerulosclerosis, 2) modification of the vascular structure, mainly in the small arterioles and 3) tubulointerstitial disease. The most characteristic aspect of diabetic nephropathy is the glomerular injury, detectable by the enlargement of the mesangium and by the thickening of the basal membrane, which often looks like a diffuse cicatrisation of the whole glomerule. The first clinical evidence of diabetic nephropathy is the presence of albuminuria or proteinuria. One refers to microalbuminuria when the amount of albumin in the urine is less than or equal to <300 mg/day and proteinuria when the total amount of protein in the urine is greater than 1 g/day.

An additional aspect of the invention concerns a method for the prevention or treatment of diabetic nephropathy in a human subject in need thereof, the method comprising administering to the human subject a compound represented by any of Formula 1, 1A, 1B and 1C as defined herein. Preferably, such administration positively affects at least one of the kidney function of the human subject. More preferably, the administration improves, maintain, or slow deterioration of at least one the kidney function. For instance, the administration may provide at least one of the following benefits: (i) slowing of the rate of the increase of proteinuria; (ii) slowing of the rate of the rise in serum creatinine; and (iii) slowing of the fall of creatinine clearance or GFR.

Typically a normal Glomerular Filtration Rate (GFR) in humans is from about 100 to about 140 mL/min. In some embodiments, the subject is a human patient having advanced nephropathy (i.e., a GFR of under 75 mL/min). In some embodiments, the subject is a human patient having ESRD (i.e., GFR of less than 10 mL/min). In some embodiments, the methods, compounds or compositions of the invention are effective in increasing the patients' GFR value by at least 1, 5, 10, 15, or 25 mL/min or more.

One of the first clinical indications of a nephropathy is the presence of albuminuria or proteinuria. One refers to microalbuminuria when the amount of albumin in the urine is less than or equal to <300 mg/day and proteinuria when the total amount of protein in the urine is greater than 1 g/day. According to some aspects, the invention relates to a method of preventing or decreasing proteinuria by administering to a subject in need thereof a compound of any of Formula 1, 1A, 1B and 1C as defined herein. In some embodiments, the subject is at risk of, or has been diagnosed with, proteinuria. In some embodiments, the subject is a human patient producing less than about 300 mg/day of protein in its urine. In some embodiments, the subject is a human patient producing more than about 1 g/day of protein in its urine. In some embodiments, the subject is a human patient having microalbuminuria. In some embodiments, the subject is a human patient with an albumin amount in the urine that exceeds 200 μg/min. In some embodiments, the methods, compounds or compositions of the invention are effective in lowering the patient's albuminuria by at least 10, 25, 50, 75, 100, 150, 200 μg/min or more.

### Other Diseases

As indicated hereinbefore, chronic hyperglycemia that is characteristic of diabetes is associated, both directly and indirectly, with a number of other diseases and unhealthy outcomes linked to increased risk of disease. Examples of such diseases include obesity, hypertension, ketone body formation and subsequent ketoacidosis, metabolic syndrome, syndrome X, dyslipidemia, hyperlipidemia, hyperlipoproteinemia, diabetic retinopathy, cardiovascular disease, atherosclerosis, thrombotic disorders, sexual dysfunction and edema. Therefore, it is reasonably anticipated by one skilled in the art that any compound which can significantly lower blood sugar concentration, such as the compounds described herein and exemplified in the present invention, may conceivably provide therapeutic benefits for diseases associated with diabetes. This is illustrated, for example, by the ability of Compound I to significantly reduce the appearance of ketone bodies in the urine and thereby prevent subsequent ketoacidosis.

### Pharmaceutical Compositions and Formulations

As indicated hereinbefore, the compounds of the invention have many potential therapeutic applications. Therefore, a related aspect of the invention concerns pharmaceutical compositions comprising a therapeutically effective amount of one or more of the compounds of the invention described herein and a pharmaceutically acceptable carrier, diluent or excipient.

As used herein, the term “pharmaceutical composition” refers to the presence of at least one compound of the invention according to Formula 1, 1A, 1B and 1C as defined herein and at least one pharmaceutically acceptable carrier, diluent, vehicle or excipient.

As used herein, the term “pharmaceutically acceptable carrier”, “pharmaceutically acceptable diluent or “pharmaceutically acceptable excipient” is intended to mean, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, or encapsulating agent, such as a liposome, cyclodextrins, encapsulating polymeric delivery systems or polyethyleneglycol matrix, which is acceptable for use in subjects, preferably humans. It preferably refers to a compound or composition that is approved or approvable by a regulatory agency of the Federal or State government or listed in the U.S. Pharmacopoeia or other generally recognized pharmacopoeia for use in animals and more particularly in humans. The pharmaceutically acceptable vehicle can be a solvent or dispersion medium containing, for example, water, ethanol, polyol (for example, glycerol, propylene glycol, and liquid polyethylene glycol), suitable mixtures thereof, and vegetable oils. Additional examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. Prevention of the action of microorganisms can be achieved by addition of antibacterial and antifungal agents, for example, parabens, chlorobutanol, phenol, ascorbic acid, thimerosal, and the like. In many cases, isotonic agents are included, for example, sugars, sodium chloride, or polyalcohols such as mannitol and sorbitol, in the composition. Prolonged absorption of injectable compositions can be brought about by including in the composition an agent which delays absorption, for example, aluminum monostearate or gelatin.

As used herein, the term “therapeutically effective amount” means the amount of compound that, when administered to a subject for treating or preventing a particular disorder, disease or condition, is sufficient to effect such treatment or prevention of that disorder, disease or condition. Dosages and therapeutically effective amounts may vary for example, depending upon a variety of factors including the activity of the specific agent employed, the age, body weight, general health, gender, and diet of the subject, the time of administration, the route of administration, the rate of excretion, and any drug combination, if applicable, the effect which the practitioner desires the compound to have upon the subject and the properties of the compounds (e.g., hydrophobicity, solubility, bioavailability, stability, potency, toxicity, etc.), and the particular disorder(s) the subject is suffering from. In addition, the therapeutically effective amount may depend on the subject's blood parameters (e.g., lipid profile, insulin levels, glycemia), the severity of the disease state, organ function, or underlying disease or complications. Such appropriate doses may be determined using any available assays including the assays described herein. When one or more of the compounds of the invention is to be administered to humans, a physician may for example, prescribe a relatively low dose at first, subsequently increasing the dose until an appropriate response is obtained.

In some embodiments, the compositions of the invention comprise a therapeutically effective amount of a compound of Formula 1, 1A, 1B and 1C. Preferred compounds are Compounds I and XIV. As indicated hereinbefore, the comp concerns pharmaceutical compositions comprising one or more of the compounds of the invention described herein (e.g., a compound of Formula I). As indicated hereinbefore, the pharmaceutical compositions of the invention may be particularly useful for subjects having type II diabetes, obesity, hyperglycemia, glucose intolerance, insulin resistance, hyperinsulinemia, hypercholesterolemia, hypertension, hyperlipoproteinemia, hyperlipidemia, hypertriglyceridemia, dyslipidemia, metabolic syndrome, syndrome X, cardiovascular disease, atherosclerosis, kidney disease, ketoacidosis, thrombotic disorders, nephropathy, diabetic neuropathy, diabetic retinopathy, sexual dysfunction, dermatopathy, dyspepsia, hypoglycemia, cerebrovascular conditions, cancer and/or edema.

In some embodiments, the invention pertains to pharmaceutical compositions that include a therapeutically effective amount of one or more compounds of Formula 1, 1A, 1B and/or 1C decreasing ketone bodies in the urine of a subject in need thereof.

In some embodiments, the invention pertains to pharmaceutical compositions that include a therapeutically effective amount of one or more compounds of Formula 1, 1A, 1B and 1C for increasing glomerular filtration rate (GFR) of a subject in need thereof.

In some embodiments, the invention pertains to pharmaceutical compositions that include a therapeutically effective amount of one or more compounds of Formula 1, 1A, 1B and/or 1C for increasing insulin secretion and/or increasing insulin sensitivity in a subject in need thereof.

In some embodiments, the invention pertains to pharmaceutical compositions that include a therapeutically effective amount of one or more compounds of Formula 1, 1A, 1B and/or 1C for decreasing insulin resistance in a subject in need thereof.

In some embodiments, the invention pertains to pharmaceutical compositions that include a therapeutically effective amount of one or more compounds of Formula 1, 1A, 1B and/or 1C for decreasing hyperglycemia in a subject in need thereof.

Preferred aspects of the invention concerns the ability of the compounds of the invention, and pharmaceutical compositions comprising same, to induce a significant, beneficial decrease in the formation of ketone bodies.

The compounds of the invention may be formulated prior to administration into pharmaceutical compositions using available techniques and procedures. For instance, the pharmaceutical compositions may be formulated in a manner suitable for administration by oral, intravenous (iv), intramuscular (im), depo-im, subcutaneous (sc), depo-sc, sublingually, intranasal, intrathecal, topical or rectal routes.

Preferably, the compound(s) of the invention can be orally administered. The formulations may conveniently be presented in unit dosage form and may be prepared by any methods well known in the art of pharmacy. Methods of preparing these formulations or compositions include the step of bringing into association a compound of the present invention with a pharmaceutically acceptable vehicle (e.g., an inert diluent or an assimilable edible carrier) and, optionally, one or more accessory ingredients. In general, the formulations are prepared by uniformly and intimately bringing into association a compound of the present invention with liquid carriers, or finely divided solid carriers, or both, and then, if necessary, shaping the product. The amount of the therapeutic agent in such therapeutically useful compositions is such that a suitable dosage will be obtained.

Formulations of the invention suitable for oral administration may be in the form of capsules (e.g., hard or soft shell gelatin capsule), cachets, pills, tablets, lozenges, powders, granules, pellets, dragees, e.g., coated (e.g., enteric coated) or uncoated, or as a solution or a suspension in an aqueous or non-aqueous liquid, or as an oil-in-water or water-in-oil liquid emulsion, or as an elixir or syrup, or as pastilles or as mouth washes and the like, each containing a predetermined amount of a compound of the present invention as an active ingredient. A compound of the present invention may also be administered as a bolus, electuary or paste, or incorporated directly into the subject's diet. Moreover, in certain embodiments these pellets can be formulated to (a) provide for instant or rapid drug release (i.e., have no coating on them); (b) be coated, e.g., to provide for sustained drug release over time; or (c) be coated with an enteric coating for better gastrointestinal tolerability. Coating may be achieved by conventional methods, typically with pH or time-dependent coatings, such that the compound(s) of the invention is released in the vicinity of the desired location, or at various times to extend the desired action. Such dosage forms typically include, but are not limited to, one or more of cellulose acetate phthalate, polyvinylacetate phthalate, hydroxypropyl methyl cellulose phthalate, ethyl cellulose, waxes, and shellac. In solid dosage forms for oral administration a compound of the present invention may be mixed with one or more pharmaceutically acceptable carriers known in the art.

Peroral compositions typically include liquid solutions, emulsions, suspensions, and the like. The pharmaceutically acceptable vehicles suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, tragacanth, and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Pharmaceutical compositions suitable for injectable use may include sterile aqueous solutions (where water soluble) or dispersions and sterile powders for the extemporaneous preparation of sterile injectable solutions or dispersions. In all cases, the composition must be sterile and must be fluid to the extent that easy syringability exists. It must be stable under the conditions of manufacture and storage and must be preserved against the contaminating action of microorganisms such as bacteria and fungi. Sterile injectable solutions can be prepared by incorporating the therapeutic agent in the required amount in an appropriate solvent with one or a combination of ingredients enumerated above, as required, followed by filtered sterilization. Generally, dispersions are prepared by incorporating the therapeutic agent into a sterile vehicle which contains a basic dispersion medium and the required other ingredients from those enumerated above. In the case of sterile powders for the preparation of sterile injectable solutions, the methods of preparation are vacuum drying and freeze-drying which yields a powder of the active ingredient (i.e., the therapeutic agent) plus any additional desired ingredient from a previously sterile-filtered solution thereof.

Some pharmaceutical formulations may be suitable for administration as an aerosol, by inhalation. These formulations comprise a solution or suspension of the desired compound of Formula 1, 1A, 1B and/or 1C as defined herein or a plurality of solid particles of such compound(s). For instance, metal salts of the compounds of this invention are expected to have physical chemical properties amenable with the preparation of fine particles of active pharmaceutical ingredient (API) for administration by inhalation but not the free acid form of these compounds. The desired formulation may be placed in a small chamber and nebulized. Nebulization may be accomplished by compressed air or by ultrasonic energy to form a plurality of liquid droplets or solid particles comprising the agents or salts. The liquid droplets or solid particles should have a particle size in the range of about 0.5 to about 5 microns. The solid particles can be obtained by processing the solid agent of any compound of Formula 1, 1A, 1B and/or 1C described herein, or a salt thereof, in any appropriate manner known in the art, such as by micronization. The size of the solid particles or droplets will be, for example, from about 1 to about 2 microns. In this respect, commercial nebulizers are available to achieve this purpose. A pharmaceutical formulation suitable for administration as an aerosol may be in the form of a liquid, the formulation will comprise a water-soluble agent of any Formula described herein, or a salt thereof, in a carrier which comprises water. A surfactant may be present which lowers the surface tension of the formulation sufficiently to result in the formation of droplets within the desired size range when subjected to nebulization.

The compositions of this invention may also be administered topically to a subject, e.g., by the direct laying on or spreading of the composition on the epidermal or epithelial tissue of the subject, or transdermally via a “patch”. Such compositions include, for example, lotions, creams, solutions, gels and solids. These topical compositions may comprise an effective amount, usually at least about 0.1%, or even from about 1% to about 5%, of a compound of the invention. Suitable carriers for topical administration typically remain in place on the skin as a continuous film, and resist being removed by perspiration or immersion in water. Generally, the carrier is organic in nature and capable of having dispersed or dissolved therein the therapeutic agent. The carrier may include pharmaceutically acceptable emollients, emulsifiers, thickening agents, solvents and the like.

Other compositions useful for attaining systemic delivery of the subject agents may include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

The compound(s) of the invention may also be administered parenterally or intraperitoneally. For such compositions, the compound(s) of the invention can be prepared in glycerol, liquid polyethylene glycols, and mixtures thereof and in oils. Under ordinary conditions of storage and use, these preparations may contain a preservative to prevent the growth of microorganisms.

### Co-Administration

While it is within the scope of the invention to use the compounds defined herein as a monotherapy, these compounds can be used in combination with other techniques (e.g., diet) and/or in combination with existing agents (e.g., antidiabetic drugs).

Accordingly, the method and compositions of the present invention may also include co-administration of the at least one compound of Formula 1, I1A, 1B and 1C as defined herein, together with the administration of another therapeutically effective agent for the prevention and/or treatment of disorders and conditions associated with abnormal levels of glucose, insulin, ketone bodies, plasma lipoprotein and/or triglycerides.

Examples of anti-diabetic agents which may be used in combination with the compounds of the present invention include insulin (injection, inhaled, short-acting, long-acting, intermediate-acting, rapid-acting, premixed), insulin secretagogues (sulfonylurea, meglitinides), alpha-glucosidase inhibitors, incretin agent, TZDs and antiobesity agents.

In one embodiment, the compound(s) of the invention is used in combination with a second therapeutic agent for lowering or controlling blood glucose level which is at least one additional known compound which is currently being used or is in development for preventing or treating diabetes. Examples of such known compounds include but are not limited to common anti-diabetic drugs such as sulphonylureas (e.g., glicazide, glipizide), metformin, glitazones (e.g., rosiglitazone, pioglitazone), prandial glucose releasing agents (e.g., repaglinide, nateglinide) and acarbose. A more detailed but non-limitative list of useful antidiabetic compounds or agents that can be used in combination with the compound(s) of the invention include insulin, biguanides, such as, for example metformin (Glucophage®, Bristol-Myers Squibb Company, U.S.; Stagid®, Lipha Sante, Europe); sulfonylurea drugs, such as, for example, gliclazide (Diamicron®), glibenclamide, gilpizide (Glucotrot® and Glucotrol XL®, Pfizer), glimepiride (Amaryl®, Aventis), chlorpropamide (e.g., Diabinese®, Pfizer), tolbutamide, and glyburide (e.g., Micronase®, Glynase®, and Diabeta®); glinides, such as, for example, repaglinide (Prandin® or NovoNorm®; Novo Nordisk), ormitiglinide, nateglinide (Starlix®), senaglinide, and BTS-67582; DPP-IV inhibitors such as vildagliptin and sitagliptin; insulin sensitizing agents, such as, for example, glitazones, a thiazolidinedione such as rosiglitazone maleate (Avandia®, Glaxo SmithKline), pioglitazone (Actos®, Eli Lilly, Takeda), troglitazone, ciglitazone, isaglitazone, darglitazone, englitazone; glucagon-like peptide I (GLP-1) receptor agonists, such as, for example, Exendin-4 (1-39) (Ex-4), Byetta™ (Amylin Pharmaceuticals Inc.), CJC-1 131 (Conjuchem Inc.), NN-221 I (Scios Inc.), GLP-1 agonists as those described in WO 98/08871; agents that slow down carbohydrate absorption, such as, for example, a-glucosidase inhibitors (e.g., acarbose, miglitol, voglibose, and emiglltate); agents that inhibit gastric emptying, such as, for example, glucagon-like peptide 1, cholescystokinin, amylin, and pramlintide; glucagon antagonists, such as, for example, quinoxaline derivatives (e.g., 2-styryl-3-[3-(dimethylamino)propylmethylamino-1-6, 7-dichloroquinoxaline, Collins et al., Bioorganic and Medicinal Chemistiy Letters 2(9):91 5-918, 1992), skyrin and skyrin analogs (e.g., those described in WO 94/14426), 1-phenyl pyrazole derivatives (e.g., those described in U.S. Pat. No. 4,359,474), substituted disllacyclohexanes (e.g., those described in U.S. Pat. No. 4,374,130), substituted pyridines and biphenyls (e.g., those described in WO 98/04528), substituted pyridyl pyrroles (e.g., those described in U.S. Pat. No. 5,776,954), 2,4-diaryl-5-pyridylimidazoles (e.g., those described in WO 98/21957, WO 98/22108, WO 98/22109, and U.S. Pat. No. 5,880,139), 2,5-substituted aryl pyrroles (e.g., those described in WO 97/1 6442 and U.S. Pat. No. 5,837,719), substituted pyrimidinone, pyridone, and pyrimidine compounds (e.g., those described in WO 98/24780, WO 98/24782, WO 99/24404, and WO 99/32448), 2-(benzimidazol-2-ylthio)-1-(3,4-dihydroxyphenyl)-1-ethanones (see Madsen et al., J. Med. Chem. 41:5151-5157, 1998), alkylidene hydrazides (e.g., those described in WO 99/01423 and WO 00/39088), glucokinase activators, such as, for example, those described in WO 00/58293, WO 01/44216, WO 01/83465, WO 01/83478, WO 01/85706, and WO 01/85707 and other compounds, such as selective ADP-sensitive K⁺ channels activators (e.g., diazoxide), hormones (e.g., cholecytokinin, GRP-bombesin, and gastrin plus EGF receptor ligands; see Banerjee et al. Rev Diabet Stud, 2005 2(3): 165-176); peroxisome proliferator-activated receptor-gamma (PPAR-gamma) agonist (e.g., pioglitazone; see Ishida et al., Metabolism, 2004, 53(4), 488-94); antioxidants (e.g., 1-bis-o-hydroxycinnamoylmethane, curcuminoid bis-demethoxycurcumin; see Srivivasan et al., J Pharm Pharm Sci. 2003, 6(3): 327-33), WO 00/69810, WO 02/00612, WO 02/40444, WO 02/40445, WO 02140446, and the compounds described in WO 97/41097 (DRF-2344), WO 97/41119, WO 97/41120, WO 98/45292, WO 99/19313 (NN622/DRF-2725), WO 00/23415, WO 00/23416, WO 00/23417, WO 00/23425, WO 00/23445, WO 00/23451, WO 00/41121, WO 00/50414, WO 00/63153, WO 00/63189, WO 00/63190, WO 00/63191, WO 00/63192, WO 00/63193, WO 00/63196, WO 00/63209, U.S. Pat. No. 6,967,019, U.S. Pat. No. 7,101,845, U.S. Pat. No. 7,074,433, U.S. Pat. No. 6,992,060, U.S. Pat. No. RE39,062, WO 2006/131836; WO 2006/120574, WO 2004/1076276, WO 2004/041266, WO 2005/086661; EP 1 630 152, EP 1 559 422, U.S. No. 2004/0038126, US No. 2006/004012, WO 2010/127440 U.S. No. 2007/0066647; and the compounds referred to in the public domain as T-174, GI-262570, YM-440, MCC-555, JTT-501, AR-H039242, KRP-297, GW-409544, CRE-16336, AR-H049020, LY510929, MBX-102, CLX-0940, and GW-501516.

Additional examples of agents that could be co-administered with the compound(s) according to the invention are compounds for stimulating pancreatic beta-cell neogenesis and/or regeneration of islets. Examples of compounds currently used or in development which have a positive effect on islet number (i.e. beta-cells) include Byetta™ (exendin-4 inhibitor), vildagliptin (Galvus™ dipeptidylpeptidase inhibitor), Januvia™ (sitagliptin phosphate) and extracts from Gymnema sylvestrae leaf (Pharma Terra). The compound(s) according to the invention may also be administered with biomolecules related to cell regeneration such as β-cellulin, plant extracts from Beta vulgaris or Ephedra herba, and nicotinamide (see Banerjee et al. Rev Diabet Stud, 2005 2(3): 165-176).

Additional compounds or agents that may be used in accordance with the principles of the present invention are those capable of inducing pancreatic beta-cell growth or insulin producing cell growth and/or insulin production. Such compounds include, but are not limited to: glucagon-like peptide-1 (GLP-1) and long-acting, DPP-IV-resistant GLP-1 analogs thereof, GLP-1 receptor agonists, gastric inhibitory polypeptide (GIP) and analogs thereof (e.g., which are disclosed in U.S. Patent Publication No. 2005/0233969), dipeptidyl peptidase IV (DPP-IV) inhibitors, insulin preparations, insulin derivatives, insulin-like agonists, insulin secretagogues, insulin sensitizers, biguanides, gluconeogenesis inhibitors, sugar absorption inhibitors, renal glucose re-uptake inhibitors, β3 adrenergic receptor agonists, aldose reductase inhibitors, advanced glycation end products production inhibitors, glycogen synthase kinase-3 inhibitors, glycogen phosphorylase inhibitors, antilipemic agents, anorexic agents, lipase inhibitors, antihypertensive agents, peripheral circulation improving agents, antioxidants, diabetic neuropathy therapeutic agents, and the like.

Additional examples of agents that may be co-administered with the compound(s) according to the invention are anti-obesity agents, and appetite reducers. Examples of anti-obesity agents that can be used with the compounds according to the invention include Xenical™ (Roche), Meridian™ (Abbott), Acomplia™ (Sanofi-Aventis), and sympathomimetic phentermine. A non-limitative list of potentially useful known and emerging anti-obesity agents is set forth in Table 2 of WO 2006/131836, that table being incorporated herein by reference.

In one embodiment, the compound(s) of the invention is used in combination with at least one additional known compound which is currently being used or in development for preventing or treating renal disorder such as nephropathy, or an associated disorder or complication. Examples of such known compounds include but are not limited to: ACE inhibitor drugs (e.g., captopril (Capoten®), enalapril (Innovace®), fosinopril (Staril®), lisinopril (Zestril®), perindopril (Coversyl®), quinapril (Accupro®), trandanalopril (Gopten®), lotensin, moexipril, ramipril); RAS blockers; angiotensin receptor blockers (ARBs) (e.g., Olmesartan, Irbesartan, Losartan, Valsartan, candesartan, eprosartan, telmisartan, etc); protein kinase C(PKC) inhibitors (e.g., ruboxistaurin); inhibitors of AGE-dependent pathways (e.g., aminoguanidine, ALT-946, pyrodoxamine (pyrododorin), OPB-9295, alagebrium); anti-inflammatory agents (e.g., cyclooxygenase-2 inhibitors, mycophenolate mophetil, mizoribine, pentoxifylline), GAGs (e.g., sulodexide (U.S. Pat. No. 5,496,807)); pyridoxamine (U.S. Pat. No. 7,030,146); endothelin antagonists (e.g., SPP 301), COX-2 inhibitors, PPAR-γ antagonists and other compounds like amifostine (used for cisplatin nephropathy), captopril (used for diabetic nephropathy), cyclophosphamide (used for idiopathic membranous nephropathy), sodium thiosulfate (used for cisplatin nephropathy), tranilast, etc. (Williams and Tuttle (2005), Advances in Chronic Kidney Disease, 12 (2):212-222; Giunti et al. (2006), Minerva Medica, 97:241-62).

Additionally, the methods of the invention may also include co-administration of at least one other therapeutic agent for the treatment of another disease directly or indirectly related to diabetes and/or renal disorder complications, including but not limited to: dyslipidemia, hypertension, obesity, neuropathy, inflammation, and/or retinopathy, etc. Additional examples of agents that can be co-administered with the compound(s) according to the invention are corticosteroids; immunosuppressive medications; antibiotics; antihypertensive and diuretic medications (such as ACE-inhibitors); lipid lowering agents such as bile sequestrant resins, cholestyramine, colestipol, nicotinic acid, and more particularly drugs and medications used to reduce cholesterol and triglycerides (e.g., fibrates (e.g., Gemfibrozil®) and HMG-CoA inhibitors such as Lovastatin®, Atorvastatin®, Fluvastatin®, Lescol®, Lipitor®, Mevacor®, Pravachol®, Pravastatin®, Simvastatin®, Zocor®, Cerivastatin®, etc); compounds that inhibit intestinal absorption of lipids (e.g., ezetiminde); nicotinic acid; and Vitamin D.

Additional examples of agents that can be co-administered with the compound(s) according to the invention are immunomodulating agents or immunosuppressants such as those that are used by type 1 diabetics who have received a pancreas transplant and/or kidney transplant (when they have developed diabetic nephropathy) (see Vinik Al at al. Advances in diabetes for the millennium: toward a cure for diabetes. Med Gen Med 2004, 6:12).

Therefore, an additional aspect of the invention relates to methods of concomitant therapeutic treatment of a subject, comprising administering to a subject in need thereof an effective amount of a first agent and a second agent, wherein the first agent is as defined in Formula I, and the second agent is for the prevention or treatment of any one of disorder or disease indicated hereinbefore. As used herein, the term “concomitant” or “concomitantly” as in the phrases “concomitant therapeutic treatment” or “concomitantly with” includes administering a first agent in the presence of a second agent. A concomitant therapeutic treatment method includes methods in which the first, second, third or additional agents are co-administered. A concomitant therapeutic treatment method also includes methods in which the first or additional agents are administered in the presence of a second or additional agent(s), wherein the second or additional agent(s), for example, may have been previously administered. A concomitant therapeutic treatment method may be executed step-wise by different actors. For example, one actor may administer to a subject a first agent and as a second actor may administer to the subject a second agent and the administering steps may be executed at the same time, or nearly the same time, or at distant times, so long as the first agent (and/or additional agents) are after administration in the presence of the second agent (and/or additional agents). The actor and the subject may be the same entity (e.g., a human).

Accordingly, the invention also relates to a method for preventing, reducing or eliminating a symptom or complication of any one of the above-mentioned diseases or conditions (e.g., diabetes, diabetic nephropathy, etc). The method comprises administering, to a subject in need thereof, a first pharmaceutical composition comprising at least one compound of the invention and a second pharmaceutical composition comprising one or more additional active ingredients, wherein all active ingredients are administered in an amount sufficient to inhibit, reduce, or eliminate one or more symptoms or complications of the disease or condition to be treated. In one aspect, the administration of the first and second pharmaceutical composition is temporally spaced apart by at least about two minutes. Preferably the first agent is a compound of Formula I as defined herein, or a pharmaceutically acceptable salt thereof, e.g., sodium salt. The second agent may be selected from the list of compounds given hereinbefore.

### Kits

The compound(s) of the invention may be packaged as part of a kit, optionally including a container (e.g., packaging, a box, a vial, etc.). The kit may be commercially used according to the methods described herein and may include instructions for use in a method of the invention. Additional kit components may include acids, bases, buffering agents, inorganic salts, solvents, antioxidants, preservatives, or metal chelators. The additional kit components are present as pure compositions, or as aqueous or organic solutions that incorporate one or more additional kit components. Any or all of the kit components optionally further comprise buffers.

The compound(s) of the invention may or may not be administered to a patient at the same time or by the same route of administration. Therefore, the methods of the invention encompass kits which, when used by the medical practitioner, can simplify the administration of appropriate amounts of two or more active ingredients to a patient.

A typical kit of the invention comprises a unit dosage form of at least one compound according to the invention, e.g., a compound Formula 1, 1A, 1B and 1C as defined herein and a unit dosage form of at least one additional active ingredient. Examples of additional active ingredients that may be used in conjunction with the compounds of the invention include, but are not limited to, any of the compounds that could be used in combination with the compound(s) of the invention as indicated hereinbefore in the section “Co-administration”.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles are provided hereinbefore.

### Assessment of Pancreas Function, Renal Function and Lipid Profiles

Quantitative assessment of pancreatic function and parameters of pancreatic diseases, pancreatic dysfunctions or pancreatic insufficiencies are well known in the art. Examples of assays for the determination of pancreas function/dysfunction includes evaluating at least one pancreatic function as assessed using biological and/or physiological parameters such as islets of Langerhans size, growth and/or secreting activity, beta-cells size, growth and/or secreting activity; insulin secretion and circulating blood levels, glucose blood levels, imaging of the pancreas, and pancreas biopsy. For instance, the examples in U.S. Pat. No. 5,424,286 describe methods for testing a compound stimulation of pancreatic insulin secretion, for testing a compound insulinotropic activity or for testing a compound activity on glycemia. Briscoe et al. (British Journal of Pharmacology, 2006, 148:619-628) disclose an insulin secretion assay using MIN6 cells.

Quantitative assessment of renal function and parameters of renal dysfunction are well known in the art and can be found, for example, in Levey (Am J Kidney Dis. 1993, 22(1):207-214). Effectiveness of the compounds according to the invention may be confirm by a reduction in the undesired symptoms which may be determined, for example, by the improvement in renal function as compared to the function prior to treatment. Such remediation may be evident in a delay in the onset of renal failure (including dialysis or transplant) or in a decrease in the rate of the deterioration of renal functions as determined for example by the slowing of the rate of the increase of proteinuria or slowing the rate of the rise in serum creatinine or by the fall in the parameter of creatinine clearance or GFR), or decrease in at least one symptom or complication including hospitalization rate or mortality. Examples of assays for the determination of renal function/dysfunction are: serum creatinine level; creatinine clearance rate; cystatin C clearance rate, 24-h urinary creatinine clearance, 24-h urinary protein secretion; Glomerular filtration rate (GFR); urinary albumin creatinine ratio (ACR); albumin excretion rate (AER); and renal biopsy.

The compounds of the invention may be tested for activity in animal models. Examples of animals models of Type II diabetes and obesity include but are not limited to: the Ob/Ob mouse (monogenic model of obesity, leptin deficient), the db/db mouse (monogenic model of obesity, leptin resistant), the Zucker (fa/fa) rat (monogenic model of obesity, leptin resistant), the Goto-Kakizaki rat, the KK mouse, the NSY mouse, the OLETF rat, the Israeli sand rat, the Fat-fed streptozotocin-treated rat, the CBA/Ca mouse, the Diabetic Torri rat, the New Zealand obese mouse (see Rees and Alcolado (2005), Diabet. Med. 22, 359-370), the NOD Mouse and its related strains, the BB Rat, leptin or leptin receptor mutant rodents, and Obese Spontaneously Hypertensive Rat (SHROB, Koletsky Rat).

Known animal models of spontaneous Type II diabetic nephropathy include: the spontaneously hypertensive/NIH-corpulent (SHR/N-cp) rat (model of obesity, Type II diabetes and nephropathy), the lean SHR/N-cp rat and the Wistar-Kyoto/NIH-corpulent (WKY/N-cp) rat (both allow assessment of the role of hypertension and obesity in the pathogenesis of diabetic nephropathy: the SHR/N-cp rats have abnormal glucose tolerance, hypertension, and develop a renal disease reminiscent of human diabetic nephropathy, whereas the WKY/N-cp rats are also obese and have hyperlipidaemia, but their glucose control is somewhat worse than that of the SHR/N-cp rat), and the LA/N-cp rat (also carries the gene for obesity, and exhibits hyperlipidaemia) (see Kimmel et al. (1992), Acta Diabetologica, Volume 29 (3-4), 142-148.

Normal levels of glucose, insulin, ketone bodies, plasma lipoprotein and/or triglycerides in human subjects are well known in the art and quantitative assessment of these biological parameters may also be useful for identifying subjects in need of treatments. Well known techniques commonly used by practitioners include measurement of fasting plasma glucose level and of plasma glucose levels in a glucose tolerance test, quantifying insulin resistance using the “hyperinsulinemic euglycemic clamp”, testing bloodstream triglyceride levels after fasting 8 to 12 h, etc.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto. The contents of all references, issued patents, and published patent applications cited throughout this application are hereby incorporated by reference. The invention is further illustrated by the following examples, which should not be construed as further limiting.

### EXAMPLES

The Examples set forth herein below provide certain representative compounds of the invention. Also provided are exemplary methods for assaying the compounds of the invention. These examples further illustrate the practice of this invention but are not intended to be limiting thereof.

Compounds I to XIII (substituted phenylacetic acid derivatives) can be synthesized according to international PCT patent publication WO 2010/127440. However, representative examples of Compounds XIV to XXXIV (substituted octanoylphenyl derivatives) are described below:

### Example 1

### Compound XIV, Sodium (RS)-2-[4-octanoylphenoxy]decanoate

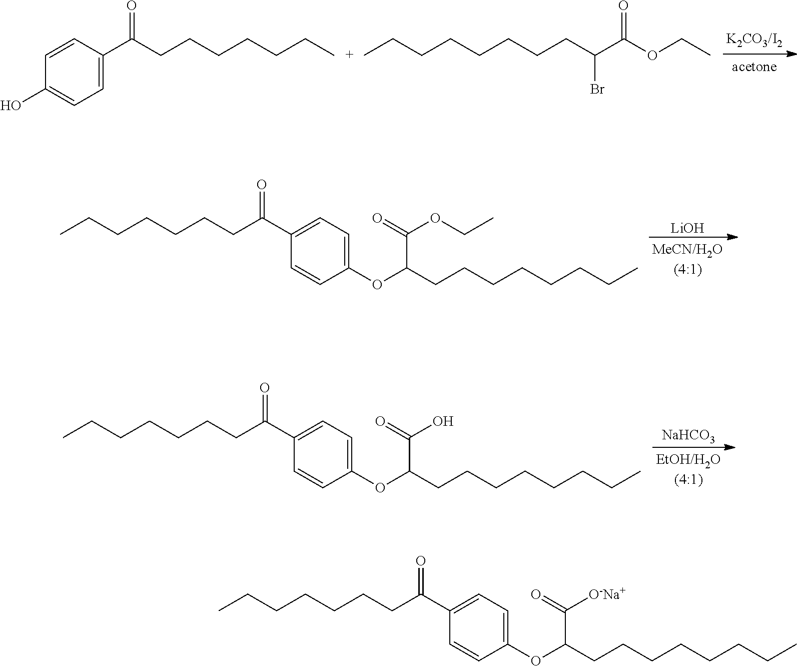

A mixture of 1-[4-hydroxyphenyl]octan-1-one (10.0 g, 45.4 mmol), K₂CO₃ (9.4 g, 68.1 mmol) and iodine (1.5 g, 9.1 mmol) in acetone (100 mL), was treated with ethyl 2-bromodecanoate (13.9 g, 49.9 mmol), and the reaction was stirred at room temperature, under nitrogen, overnight. Solvent was evaporated in vacuo, and the residue was partitioned between ethyl acetate and water. The organic phase was washed with saturated aqueous sodium chloride, dried over magnesium sulfate, filtered and evaporated in vacuo. The crude material was purified on a silica gel pad, eluted with 5% ethyl acetate/hexane to give ethyl (RS)-2-[4-octanoylphenoxy]decanoate (11.9 g, 62%) as a colourless oil. ¹H NMR (400 MHz, CDCl₃): δ 7.92 (d, J=9.0 Hz, 2H), 6.89 (d, J=9.0 Hz, 2H), 4.66 (dd, J=7.5, 5.2 Hz, 1H), 4.21 (q, J=7.0 Hz, 2H), 2.89 (t, J=7.4 Hz, 2H), 1.90-2.03 (m, 2H), 1.66-1.74 (m, 2H), 1.43-1.56 (m, 2H), 1.24-1.37 (m, 18H), 1.24 (t, J=7.2 Hz, 2H), 0.85-0.89 (m, 6H). A solution of ethyl ester (11.9 g, 28.3 mmol) in a mixture of tetrahydrofuran (360 mL), methanol (90 mL) and water (90 mL), was treated with lithium hydroxide monohydrate (5.9 g, 141.5 mmol), and the mixture was stirred at room temperature for 20 hours. A second portion of lithium hydroxide monohydrate (2.3 g, 54.8 mmol) was added and the reaction was stirred at room temperature for an additional 3 hours. The reaction mixture was concentrated in vacuo and the residue was partitioned between ethyl acetate and water. The organic phase was washed with saturated aqueous sodium chloride, dried over magnesium sulfate, filtered and evaporated in vacuo, to give the crude product. Purification on a silica gel pad, eluted with 40% ethyl acetate/hexane; and recrystallization from hexanes gave (RS)-2-[4-octanoylphenoxy]decanoic acid (9.46 g, 86%) as a white solid. m.p. 45-47° C.; ¹H NMR (400 MHz, CDCl₃): δ 7.93 (d, J=9.0 Hz, 2H), 6.91 (d, J=9.0 Hz, 2H), 4.72 (dd, J=6.8, 5.7 Hz, 1H), 2.90 (t, J=7.4 Hz, 2H), 1.98-2.04 (m, 2H), 1.67-1.74 (m, 2H), 1.46-1.59 (m, 2H), 1.24-1.37 (m, 18H), 0.87 (t, J=6.9 Hz, 3H), 0.88 (t, J=6.9 Hz, 3H). A solution of the acid (9.4 g, 24.1 mmol) in ethanol (200 mL) was treated with a solution of sodium bicarbonate (2.0 g, 24.1 mmol) in water (50 mL), and the reaction was stirred at room temperature for 5 hours. Solvents were concentrated in vacuo, and the solution was diluted with water (950 mL), filtered (0.2 μm), and lyophilised to give sodium (RS)-2-[4-octanoylphenoxy]decanoate as a white solid (8.8 g, 88%). mp 275-280° C.; ¹H NMR (400 MHz, CD₃OD): δ 7.96 (d, J=9.0 Hz, 2H), 6.97 (d, J=9.0 Hz, 2H), 4.72 (dd, J=6.2, 5.9 Hz, 1H), 2.95 (t, J=7.4 Hz, 2H), 1.94-1.99 (m, 2H), 1.64-1.72 (m, 2H), 1.49-1.57 (m, 2H), 1.28-1.40 (m, 18H), 0.90 (t, J=6.9 Hz, 3H), 0.89 (t, J=6.9 Hz, 3H); ¹³C NMR (101 MHz, CD₃OD): δ 200.72, 177.83, 163.37, 130.20, 129.61, 114.70, 79.55, 37.94, 33.19, 31.87, 31.76, 29.45, 29.38, 29.24, 29.22, 29.16, 25.74, 24.85, 22.57, 22.52, 13.29, 13.28; LRMS (ESI): m/z 391 (M−Na⁺+2H⁺); HPLC: 6 min.

Resolution of the enantiomers of compound XIV.

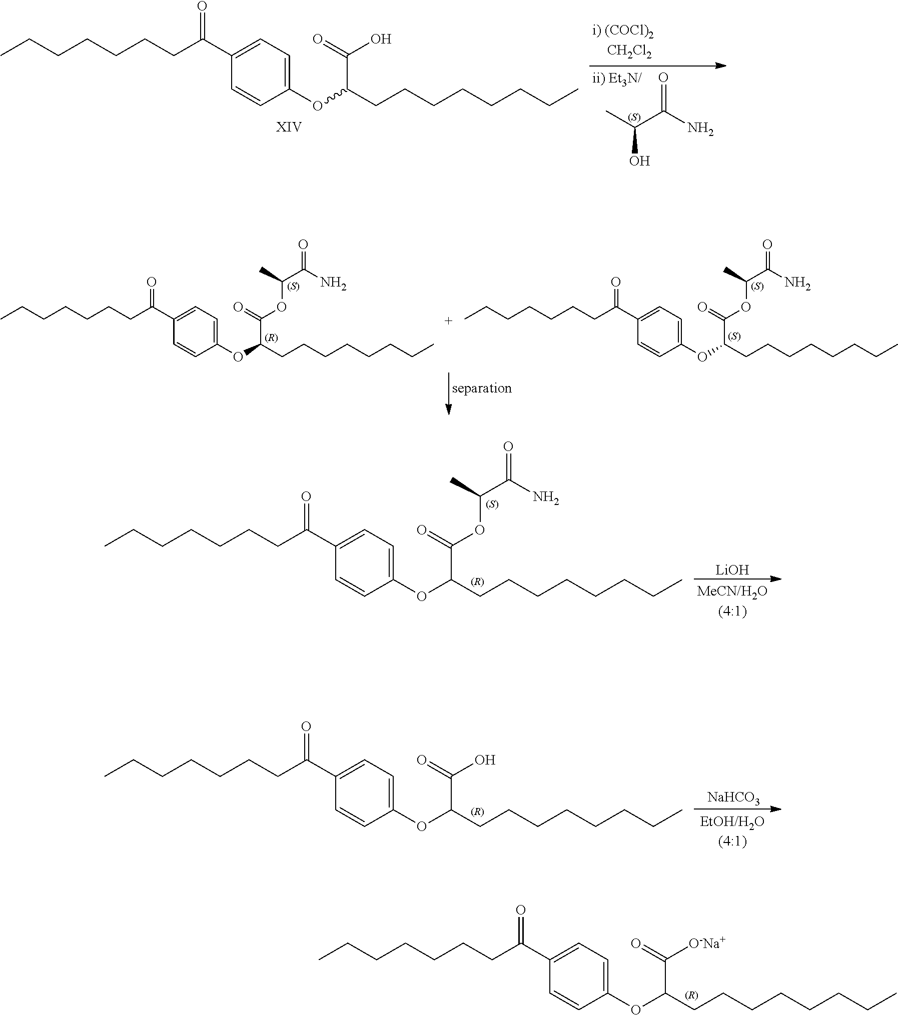

The same procedure was repeated for the (S) isomer

Sodium Salts of (R)- & (S)-2-[4-Octanoylphenoxy]decanoate

1) Formation and Separation of (S)-Lactamide Esters:

A solution of (RS)-2-[4-octanoylphenoxy]decanoic acid (0.95 g, 2.4 mL) in dichloromethane (20 mL) was treated dropwise with oxalyl chloride (0.26 mL, 3.1 mmol), and the reaction was stirred at room temperature for 1 hour. Triethylamine (0.51 mL, 3.7 mmol) was added, followed by (S)-lactamide (0.54 g, 6.1 mmol), and the reaction was stirred at room temperature for 20 hours. The solution was then diluted with ethyl acetate (100 mL), and washed with 1M aqueous HCl (100 mL), water (100 mL) and saturated aqueous sodium chloride (50 mL), then dried over sodium sulphate and evaporated in vacuo. The two diastereomers were separated on a Biotage™ 40L column (silica), eluted with diethyl ether/hexane 1:4 to 1:1, then with ethyl acetate/hexane 1:4 to 1:1. This give the separate pure diastereomers.

First diastereomer (0.51 g, 45%) as a white, waxy solid:

¹H NMR (400 MHz, CDCl₃): δ 7.93 (d, J=9.0 Hz, 2H), 6.91 (d, J=8.8 Hz, 2H), 5.68 (br s, 1H), 5.54 (br s, 1H), 5.22 (q, J=6.8 Hz, 1H), 4.77 (dd, J=7.3, 5.2 Hz, 1H), 2.88 (t, J=7.5 Hz, 2H), 1.92-2.08 (m, 2H), 1.69, (tt, J=7.3, 7.3 Hz, 2H), 1.46-1.56 (m, 2H), 1.47, (d, J=6.8 Hz, 3H), 1.23-1.38 (m, 18H), 0.86 (t, J=6.6 Hz, 6H); ¹³C NMR (101 MHz, CDCl₃): δ 199.15, 172.34, 170.09, 161.35, 131.47, 130.82, 114.56, 76.70, 71.16, 38.59, 32.90, 32.00, 31.93, 29.57, 29.52, 29.35 (3C), 25.26, 24.68, 22.84 (2C), 17.85, 14.29 (2C).

Second diastereomer (0.47 g, 42%) as a viscous, colourless oil:

¹H NMR (400 MHz, CDCl₃): δ 7.90 (d, J=9.0 Hz, 2H), 6.91 (d, J=9.0 Hz, 2H), 6.25 (br s, 1H), 6.15 (br s, 1H), 5.20 (q, J=6.9 Hz, 1H), 4.79 (dd, J=6.6, 5.9 Hz, 1H), 2.88 (t, J=7.5 Hz, 2H), 1.95-2.01 (m, 2H), 1.68, (tt, J=7.3, 7.3 Hz, 2H), 1.47-1.55 (m, 2H), 1.39, (d, J=6.8 Hz, 3H), 1.22-1.37 (m, 18H), 0.86 (t, J=6.8 Hz, 6H); ¹³C NMR (101 MHz, CDCl₃): δ 199.43, 172.71, 170.29, 161.52, 131.31, 130.60, 114.84, 76.48, 71.13, 38.59, 32.80, 32.00, 31.93, 29.58, 29.53, 29.36 (3C), 25.36, 24.76, 22.84, 17.69, 14.29 (2C).

2) Conversion of Diastereomers to the Corresponding Sodium Salt:

General Procedure:

A solution of diastereomeric ester (1.73 g, 3.7 mmol) in acetonitrile (72 mL) was treated with a solution of lithium hydroxide (0.45 g, 18.7 mmol) in water (18 mL), and the reaction was stirred at room temperature for 17 hours. The reaction was quenched by addition of 1M aqueous HCl (150 mL), and extracted with ethyl acetate (2×100 mL). Combined extracts were washed with water (150 mL) and saturated aqueous sodium chloride (150 mL); then dried over sodium sulfate, filtered and evaporated in vacuo to give the crude acid.

First Enantiomer (Higher R_{f}, Silica Gel):

Purification on a Biotage™ 40L column (silica), eluted with ethyl acetate/hexane 1:9 to 7:3, gave the purified acid enantiomer as a white solid (1.28 g, 87%). ¹H NMR (400 MHz, CDCl₃): δ 11.50 (s, 1H), 7.92 (d, J=8.8 Hz, 2H), 6.90 (d, J=9.0 Hz, 2H), 4.71 (dd, J=6.4, 5.9 Hz, 1H), 2.89 (t, J=7.4 Hz, 2H), 1.97-2.03 (m, 2H), 1.69, (tt, J=7.1, 7.1 Hz, 2H), 1.45-1.59 (m, 2H), 1.21-1.38 (m, 18H), 0.862 (t, J=7.0 Hz, 3H), 0.859 (t, J=6.8 Hz, 3H); ¹³C NMR (101 MHz, CDCl₃): δ 200.20, 176.59, 161.76, 131.00, 130.77, 114.83, 76.15, 38.59, 32.80, 32.03, 31.93, 29.59, 29.53, 29.39, 29.37 (2C), 25.38, 24.91, 22.89 (2C), 14.30 (2C).

A solution of the acid (1.28 g, 3.2 mmol) in ethanol (20 mL) was treated with a solution of sodium bicarbonate (0.27 g, 3.2 mmol) in water (5 mL), and the reaction was stirred at room temperature for 3 days. Solvents were evaporated in vacuo to give the crude salt as a white waxy solid. This material was dissolved in water (130 mL), filtered (0.2 micron; nylon) and lyophilised to give the pure enantiomer as a white solid (1.1 g, 97%). ¹H NMR (400 MHz, CD₃OD): δ 7.91 (d, J=8.6 Hz, 2H), 6.96 (d, J=8.8 Hz, 2H), 4.46 (t, J=6.2 Hz, 1H), 2.92 (t, J=7.3 Hz, 2H), 1.90-1.95 (m, 2H), 1.66, (tt, J=7.2, 7.2 Hz, 2H), 1.44-1.61 (m, 2H), 1.24-1.39 (m, 18H), 0.890 (t, J=6.7 Hz, 3H), 0.882 (t, J=6.7 Hz, 3H); ¹³C NMR (101 MHz, CD₃OD): δ 200.66, 177.83, 163.37, 130.24, 129.64, 114.73, 79.59, 37.96, 33.20, 31.87, 31.76, 29.46, 29.40, 29.26, 29.22, 29.16, 25.75, 24.86, 22.57, 22.53, 13.32, 13.29; other data to be collected.

Second Enantiomer (Lower R_{f}, Silica Gel):

Purification on a Biotage™ 40L column (silica), eluted with ethyl acetate/hexane 1:9 to 7:3, gave the purified acid enantiomer as a white solid (1.10 g, 87%). ¹H NMR (400 MHz, CDCl₃): 11.51 (s, 1H), 7.91 (d, J=9.0 Hz, 2H), 6.90 (d, J=9.0 Hz, 2H), 4.71 (dd, J=6.6, 5.9 Hz, 1H), 2.89 (t, J=7.5 Hz, 2H), 1.97-2.03 (m, 2H), 1.69, (tt, J=7.1, 7.1 Hz, 2H), 1.45-1.58 (m, 2H), 1.21-1.37 (m, 18H), 0.862 (t, J=7.0 Hz, 3H), 0.858 (t, J=7.0 Hz, 3H); ¹³C NMR (101 MHz, CDCl₃): δ 200.16, 176.47, 161.77, 131.03, 130.76, 114.84, 76.18, 38.58, 32.79, 32.02, 31.93, 29.58, 29.52, 29.37, 29.36 (2C), 25.36, 24.91, 22.84 (2C), 14.35, 14.28.

A solution of the acid (1.1 g, 2.7 mmol) in ethanol (16 mL) was treated with a solution of sodium bicarbonate (0.23 g, 2.7 mmol) in water (4 mL), and the reaction was stirred at ambient temperature for 18 hours. Solvents were evaporated in vacuo to give the crude salt as a clear, colourless syrup. This material was dissolved in water (100 mL), filtered (0.2 micron; nylon) and lyophilised to give the pure enantiomer as a white solid (1.12 g, 99%). ¹H NMR (400 MHz, CD₃OD): δ 7.91 (d, J=9.0 Hz, 2H), 6.96 (d, J=9.0 Hz, 2H), 4.46 (t, J=6.2 Hz, 1H), 2.92 (t, J=7.4 Hz, 2H), 1.90-1.95 (m, 2H), 1.66, (tt, J=7.1, 7.1 Hz, 2H), 1.45-1.61 (m, 2H), 1.24-1.39 (m, 18H), 0.890 (t, J=6.8 Hz, 3H), 0.881 (t, J=6.9 Hz, 3H); ¹³C NMR (101 MHz, CD₃OD): δ 200.65, 177.82, 163.37, 130.20, 129.65, 114.74, 79.58, 37.96, 33.19, 31.87, 31.76, 29.46, 29.40, 29.26, 29.22, 29.16, 25.75, 24.86, 22.57, 22.53, 13.32, 13.29.

Compound XV, Sodium 3-octanoylbenzoate

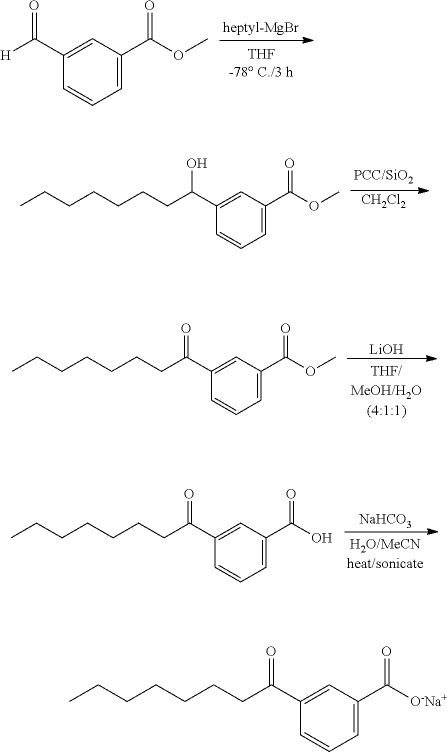

A solution of methyl 3-formylbenzoate (2.0 g, 12.2 mmol) in tetrahydrofuran (40 mL) was cooled to −78° C. under nitrogen. A solution of n-heptylmagnesium bromide in tetrahydrofuran (1 M; 12.2 mL, 12.2 mmol) was added dropwise over 30 min, and the reaction was stirred at −78° C. for 3 hours. The reaction was quenched by addition of aqueous HCl (1 M), and the mixture was extracted (×3) with ethyl acetate. Extracts were combined, dried over sodium sulfate, filtered and evaporated in vacuo. The crude material was purified on a Biotage™ 40 M column (silica), eluted with 10% ethyl acetate/hexane to give methyl (RS)-3-[1-hydroxyoctyl]benzoate (2.2 g, 69%) as a colourless oil. ¹H NMR (400 MHz, CDCl₃): δ 7.98 (s, 1H), 7.91 (d, J=7.8 Hz, 1H), 7.53 (d, J=7.8 Hz, 1H), 7.39 (dd, J=7.8, 7.8 Hz, 1H), 4.65-4.71 (s, 1H), 3.89 (s, 3H), 2.33 (d, J=3.1 Hz, 1H), 1.62-1.80 (m, 2H), 1.18-1.41 (m, 10H), 0.85 (t, J=6.9 Hz, 3H). A solution of the secondary alcohol (2.0 g, 7.5 mmol) in dichloromethane (50 mL) was treated with silica gel (16 g) and pyridinium chlorochromate (3.2 g, 15.0 mmol), and the reaction was stirred at room temperature overnight. The reaction mixture was filtered through silica gel, and the residue was washed with dichloromethane. Combined filtrate and washings were evaporated in vacuo to give methyl 3-octanoylbenzoate (9.5 g, 86%). ¹H NMR (400 MHz, CDCl₃): δ 8.58-8.59 (m, 1H), 8.20-8.23 (m, 1H), 8.14-8.17 (m, 1H), 7.53-7.57 (m, 1H), 3.95 (s, 3H), 3.00 (t, J=7.3 Hz, 2H), 1.74 (tt, J=7.3, 7.3 Hz, 2H), 1.24-1.40 (m, 8H), 0.88 (t, J=6.9 Hz, 3H). A solution of the methyl ester (1.0 g, 3.8 mmol) in tetrahydrofuran (30 mL), was treated with a solution of lithium hydroxide monohydrate (800 mg, 19.1 mmol) in water (7 mL). Methanol (7 mL) was then added, and the mixture was stirred at room temperature for 24 hours. The reaction mixture was treated with aqueous HCl (1 M) until the pH was below 5, and was then extracted with ethyl acetate (×3). Organic extracts were combined, washed with saturated aqueous sodium chloride, dried over sodium sulfate, filtered and evaporated in vacuo, to give 3-octanoylbenzoic acid (919 mg, 97%). ¹H NMR (400 MHz, CD₃OD): δ 8.59 (dd, J=1.7, 1.2 Hz, 1H), 8.18-8.24 (m, 2H), 7.61 (ddd, J=7.8, 7.8, 0.4 Hz, 1H), 3.05 (t, J=7.3 Hz, 2H), 1.71 (tt, J=7.3, 7.3 Hz, 2H), 1.27-1.41 (m, 8H), 0.90 (t, J=7.0 Hz, 3H). A mixture of the acid (919 mg, 3.7 mmol) and sodium bicarbonate (311 mg, 3.7 mmol) was treated with water (20 mL), and the reaction was heated with sonication and stirring until most of the solids had dissolved. Acetonitrile was added and the mixture was filtered (0.45 μm), and lyophilised to give sodium 3-octanoylbenzoate as a white solid (1.0 g, 100%). ¹H NMR (400 MHz, D₂O): δ 8.14 (s, 1H), 7.81 (d, J=7.8 Hz, 1H), 7.61 (d, J=8.0 Hz, 1H), 7.18 (dd, J=8.0, 7.8 Hz, 1H), 2.69 (t, J=6.8 Hz, 2H), 1.33 (tt, J=7.0, 7.0 Hz, 2H), 0.88-1.03 (m, 8H), 0.54 (t, J=7.0 Hz, 3H). ¹³C NMR (101 MHz, D₂O): δ 203.93, 173.62, 137.25, 136.27, 133.92, 130.27, 128.59, 128.48, 38.58, 31.41, 28.82, 28.79, 24.25, 22.32, 13.60; LRMS (ESI): m/z 249 (M−Na⁺+2H⁺); HPLC: 4 min.

Compound XVI, Sodium (RS)-5-octanoyl-indane-2-carboxylate

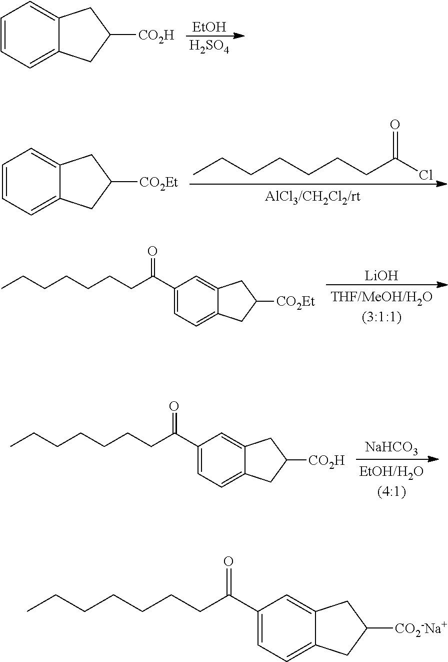

A solution of indane-2-carboxylic acid (504 mg, 3.1 mmol) and sulphuric acid (2 mL) in dry ethanol, was heated at 75° C. for 3 days. The solution was concentrated in vacuo, and then partitioned between dichloromethane and water. The pH of the aqueous layer was adjusted to 13-14 with aqueous sodium hydroxide (5 M), and the layers were separated. The aqueous phase was diluted with saturated sodium chloride, and extracted (2×) with dichloromethane. Combined organic extracts were washed with saturated sodium chloride, dried over sodium sulfate, filtered and evaporated in vacuo, to give the crude product. Purification on a Biotage™ 25S column (silica), eluted with 3% ethyl acetate/hexane, gave ethyl indane-2-carboxylate (526 mg, 96%). ¹H NMR (400 MHz, CDCl₃): δ 7.22-7.26 (m, 2H), 7.17-7.20 (m, 2H), 4.21 (q, J=7.0 Hz, 2H), 3.19-3.39 (m, 5H), 1.31 (t, J=7.0 Hz, 3H). A mixture of ethyl indane-2-carboxylate (100 mg, 0.5 mmol) and aluminum chloride (164 mg, 1.2 mmol) in dichloromethane (4 mL), was treated with octanoyl chloride (0.1 mL, 0.5 mmol) at room temperature, and the reaction was stirred at this temperature overnight. The reaction mixture was poured onto a mixture of ice and aqueous. HCl (1 M), and extracted (3×) with dichloromethane. Combined organic extracts were dried over magnesium sulfate, filtered and evaporated in vacuo. The crude material was purified on a Biotage™ column (silica), eluted with 5% ethyl acetate/hexane, to give ethyl (RS)-5-octanoyl-indane-2-carboxylate (110 mg, 65%). ¹H NMR (400 MHz, CDCl₃): δ 7.69-7.77 (m, 2H), 7.29-7.32 (m, 1H), 4.07-4.17 (m, 2H), 3.15-3.36 (m, 5H), 2.84-2.90 (m, 2H), 1.62-1.70 (m, 2H), 1.19-1.34 (m, 8H), 0.80-0.87 (m, 3H) A suspension of the ethyl ester (82 mg, 0.3 mmol) in a mixture of tetrahydrofuran (3 mL), methanol (1 mL) and water (1 mL), was treated with lithium hydroxide (43 mg, 1.8 mmol), and the mixture was stirred at room temperature overnight. The reaction mixture was concentrated in vacuo and the residue was diluted with water. The pH was adjusted to pH 4 with aqueous HCl (1 M), and the mixture was extracted (3×) with ethyl acetate. Combined organic extracts were dried over magnesium sulfate, filtered and evaporated in vacuo, to give the crude product. Purification on a Biotage™ 12 M column (silica), eluting with 2% ethyl acetate/hexane, gave (RS)-5-octanoyl-indane-2-carboxylic acid (60 mg, 80%). ¹H NMR (400 MHz, CD₃OD): δ 7.80 (s, 1H), 7.78 (dd, J=7.8, 1.4 Hz, 1H), 7.30 (d, J=7.8 Hz, 1H), 3.36 (tt, J=8.2, 8.2 Hz, 1H), 3.24 (d, J=8.2 Hz, 4H), 2.96 (t, J=7.4 Hz, 2H), 1.67 (tt, J=7.2, 7.2 Hz, 2H), 1.26-1.39 (m, 8H), 0.89 (t, J=6.9 Hz, 3H). A solution of the acid (60 mg, 0.2 mmol) in ethanol (4 mL) and water (1 mL) was treated with sodium bicarbonate (18 mg, 0.2 mmol), and the reaction was stirred at room temperature overnight. Solvents were concentrated in vacuo, and the solution was diluted with water, filtered (20 μm), and lyophilised to give sodium (RS)-5-octanoyl-indane-2-carboxylate as a white solid (54 mg, 87%). ¹H NMR (400 MHz, CD₃OD): δ 7.91 (s, 1H), 7.76 (dd, J=7.8, 1.6 Hz, 1H), 7.28 (d, J=7.8 Hz, 1H), 3.16-3.25 (m, 5H), 2.97 (t, J=7.3 Hz, 2H), 1.68 (tt, J=7.3, 7.3 Hz, 2H), 1.28-1.40 (m, 8H), 0.90 (t, J=7.0 Hz, 3H); LRMS (ESI): m/z 289 (M−Na⁺+2H⁺); HPLC: 5 min.

### Example 2

### In Vivo Effect of Compound I on Streptozotocin-Induced Diabetes in Rats

Demonstration of the in vivo effect by oral administration of Compound I was undertaken in the streptozotocin-induced diabetes model using the following procedure. Diabetes was induced by a single intraperitoneal administration of streptozotocin (65 mg/kg of body weight) in fasted male Sprague Dawley rats, weighing approximately 200-250 g. After 48 hours, rats with plasma glucose more than 10 mmole/L were enrolled and treated with vehicle or compounds.

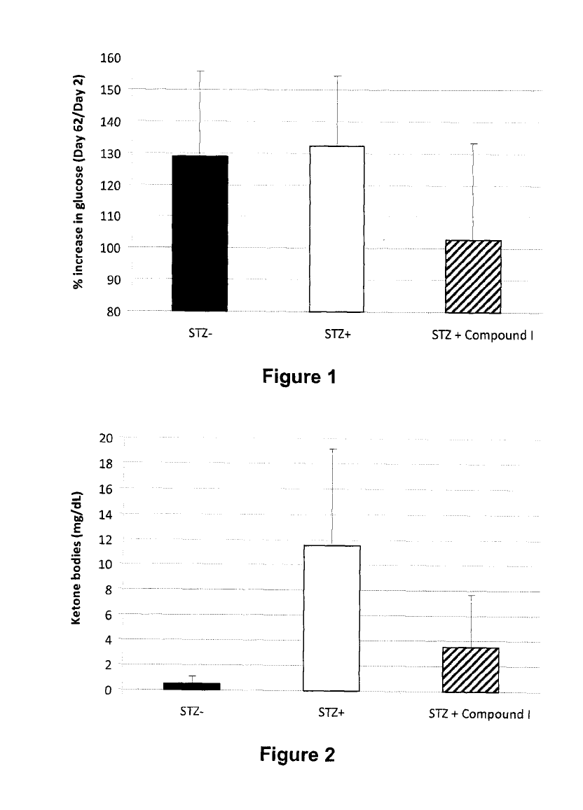

Streptozotocin is well known to induce 1-islets toxicity. As shown in FIG. 1, treatment with Compound I (100 mg/kg) decrease significantly (p<0.05) the level of increase in blood glucose (delta) from day 2 to day 62 post-streptozotocin. This result supports the role of compounds of Formula 1, 1A, 1B and 1C as defined herein in preventing and/or treating a diabetes-related disorder and/or a pancreatic disease.

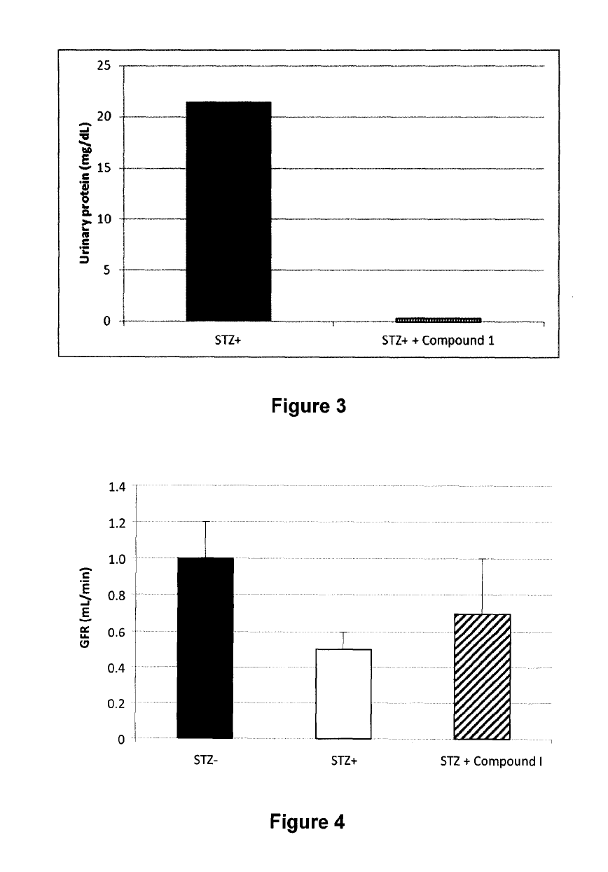

Ketonuria is a medical condition in which ketone bodies are present in the urine. It is seen in conditions in which the body produces excess ketones as an alternative source of energy. It is seen during starvation or more commonly in diabetes. In a diabetic patient, ketone bodies in the urine suggest that the patient is not adequately controlled and that adjustments of medication, diet, or both should be made promptly. As shown in FIG. 2, ketone bodies are significantly (p<0.05) reduced in urine of treated rats. This result further supports the role of compounds of Formula 1, 1A, 1B and 1C as defined herein in preventing and/or treating diabetes-related disorders and/or pancreatic diseases, and more particularly in decreasing ketone bodies in the urine of mammals.
Proteinuria is a medical condition in which an excess of protein is present in the urine. Proteinuria may be a sign of renal kidney damage. Since serum proteins are readily reabsorbed from urine, the presence of excess protein indicates either an insufficiency of absorption or impaired filtration. Diabetics may suffer from damaged nephrons and develop proteinuria. The most common cause of proteinuria is diabetes. As shown in FIG. 3, oral treatment with Compound I (100 mg/kg) completely abbrogated the presence of urinary protein that was detected in STZ+(diabetic).animals

Diabetic nephropathy is the largest single cause of end-stage renal failure. Despite the available therapies of glycemic and blood pressure control, many patients continue to show progressive renal damage. Therefore, it is extremely important to identify novel interventions to halt the progression of diabetic nephropathy. FIG. 4 represents the significant (p<0.05) improvement of glomerular filtration rate (GFR) as demonstrated by creatinine clearance in diabetic rats treated with Compound I. This result supports the role of compounds of Formula 1, 1A, 1B and 1C as defined herein in preventing and/or treating diabetic neuropathy.

### Example 3

### In Vivo Effect of Compound XIV on Glucose Concentration in the 5/6 Nephrectomized Rat Model

Demonstration of the in vivo protective effect of Compound XIV on serum glucose concentration was measured in the 5/6 nephrectomized (Nx) rat model using the following procedure. Male 6 week-old Wistar rats were subjected to 5/6 nephrectomy or sham operations. Under fluothane anesthesia, renal ablation was achieved by removing two-thirds of the left kidney followed by a right unilateral nephrectomy 7 days later. Sham rats underwent exposition of the kidneys and removal of the perirenal fat. Animals that underwent the sham operation were given vehicle (saline) and were used as controls. Nx animals were divided in groups receiving the vehicle or Compound XIV. Saline or Compound XIV was given by gastric gavage once daily up to the sacrifice. Serum glucose was measured every three weeks in order to assess the effect of the compound on serum glucose concentration in a renal disease model. Rats were sacrificed at day 126.

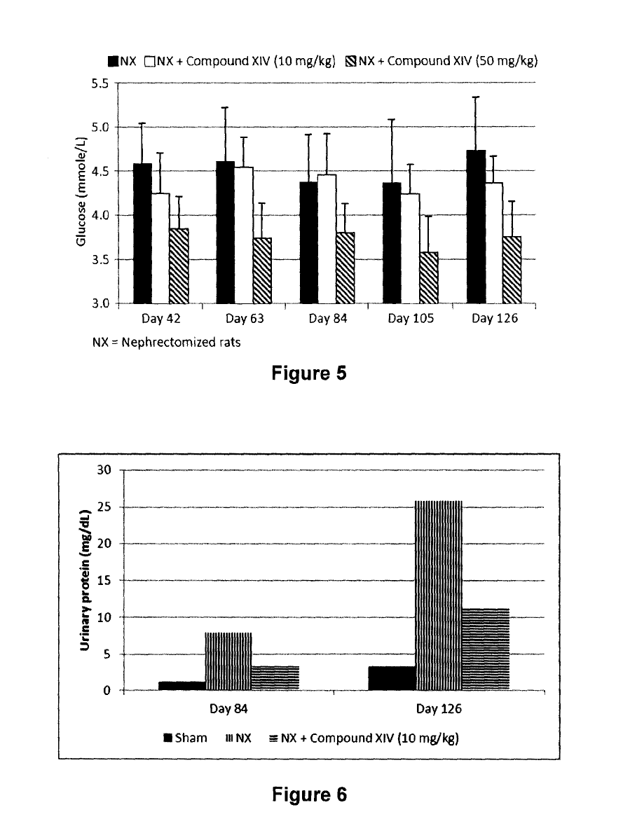

Serum glucose level varies from 4.8 to 5.2 mmole/L. FIG. 5 represents the serum glucose concentration in Nx and Compound XIV-treated Nx rats. Treatment with Compound XIV at a concentration of 50 mg/kg induces a significant (p<0.01) decrease in serum glucose from day 42 to day 126.

FIG. 6 represents the urinary proteins concentration in Nx and Compound XIV-treated Nx rats. Treatment with Compound XIV at a concentration of 10 mg/kg induces a significant (p<0.01) decrease in urine proteins at day 84 and 126.

### Example 4

### In Vivo Effect of Compound I on Glucose Concentration in Diabetic Db/Db Mice

Demonstration of the in vivo effect of Compound I was measured in the diabetic mouse (db/db) model using the following procedure. Male Lepr db (db/db) and age- and sex-matched control mice with the same genetic background (C57BL/6) were used. Animals were randomized using blood sugar measurement (5-hours starved) at week 6 and uninephrectomy (removal of right kidney) was performed. Sham animals (C57BL/6) underwent exposition of the right kidney. Animals that underwent the sham operation were given vehicle (saline) and were used as negative controls. C57BL/6 mice that were uninephrectomized were given vehicle and were used as positive controls. Db/db mice were treated with oral administration of compound I at 100 mg/kg per day for 131 days. Serum glucose was measured every two to four weeks. Oral glucose tolerance test was performed on 16-hours starved mice at day 112. Glomerular filtration rate (GFR) was determined by clearance kinetics of plasma fluorescein isothiocyanate-inulin at 5, 15, 30, 60 and 120 minutes after a single intravenous bolus injection. Fluorescence was determined and GFR calculated based on a two-compartment model using nonlinear regression curve-fitting software.

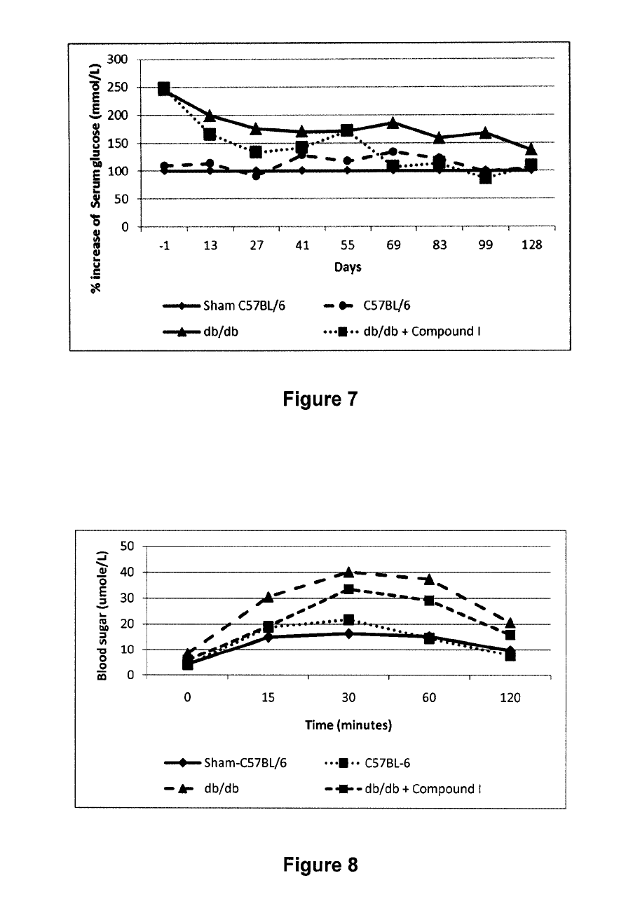

FIG. 7 represents the percent increase of serum glucose concentration of C57BL/6, db/db uninephrectomized mice and with treatment with Compound I compared to C57BL/6 sham mice (negative control, 100%). Db/db mice had a high level of blood sugar compared to C57BL/6 uninephrectomized or sham. Treatment with Compound I at a concentration of 100 mg/kg induces a significant decrease in serum glucose from day 69 to day 128.

Oral glucose tolerance test (OGTT) which measures the ability to metabolize glucose or clear it out of the bloodstream, was performed at day 112. FIG. 8 represents OGTT of non-diabetic (sham and C57BL/6 uninephrectomized mice) and diabetic (Db/db mice) treated or not with Compound I. Treatment with Compound I induces a significant increase in sugar blood clearance.

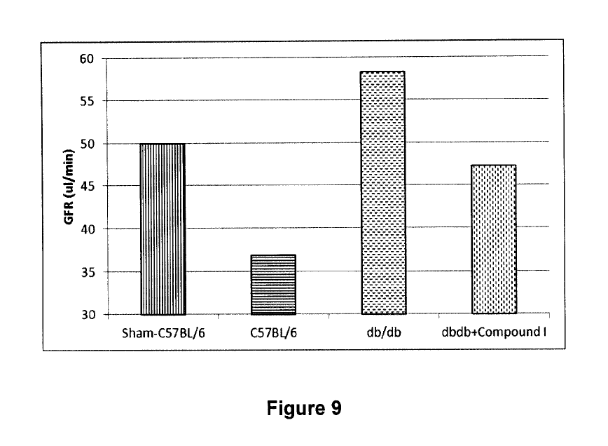

Diabetic nephropathy was measured by GFR. FIG. 9 represents the clearance of inulin which is used to determine GFR. C57BL/6 uninephrectomized mice show a significant reduction in GFR compared to Sham C57BL/6 mice. Hyperfiltration is observed in Db/db uninephrectomized mice compared to C57BL/6 uninephrectomized mice indicating diabetes. Treatment with Compound I reduces diabetes-induced hyperfiltration.

Headings are included herein for reference and to aid in locating certain sections These headings are not intended to limit the scope of the concepts described therein, and these concepts may have applicability in other sections throughout the entire specification Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1-28. (canceled)

29. A method for treating diabetes or a diabetes-related disorder, wherein said method comprises administering, to a subject in need of such treatment, a compound represented by Formula 1, or a pharmaceutically acceptable salt thereof:
Cy-Q Formula 1
wherein
Cy is 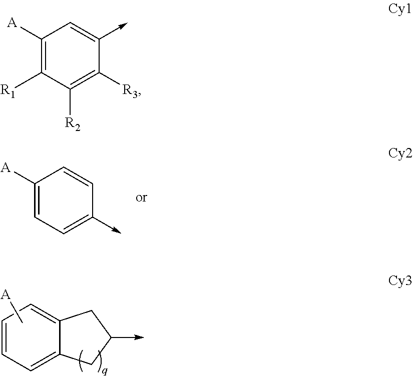
where
q is 1, 2 or 3;
→ represents a covalent bond connecting Cy to Q;
A is
1) C₁-C₆ alkyl,
2) C₂-C₆ alkenyl,
3) C₁-C₇ alkyl-Y—,
4) C₁-C₇ alkyl-OC(O)—, or
5) C₁-C₇ alkyl-CH(OH)—,
where Y is O, S or C(O);
R₁, R₂ and R₃ are independently selected from H, F, Cl and OH;
when Cy is Cy1 or Cy2, then Q is
1) C(O)OH,
2) C(CH₃)₂C(O)OH,
3) (CH₂)mC(O)OH,
4) ZCH(C(O)OH)C₁-C₈ alkyl,
5) Z(CH₂)mC(O)OH, or
6) CH₂CH(C(O)OH)C₁-C₈ alkyl,
where
Z is O or S,
m is 1 or 2; and
when Cy is Cy3, then Q is C(O)OH.

30. The method of claim 29, wherein the compound is represented by Formula 1A, or a pharmaceutically acceptable salt thereof: 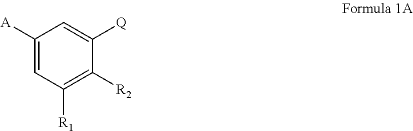
wherein
A is
1) C₁-C₆ alkyl, or
2) C₁-C₆ alkenyl;
R₁ and R₂ are independently selected from H, F, Cl and OH;
Q is
1) C(O)OH,
2) C(CH₃)₂C(O)OH, or
3) (CH₂)mC(O)OH,
where m is 1 or 2.

31. The method of claim 29, wherein the compound is represented by Formula 1B, or a pharmaceutically acceptable salt thereof: 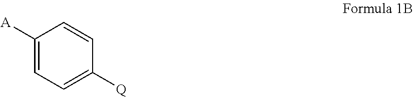
wherein
A is
1) C₁-C₆ alkyl, or
2) C₁-C₆ alkenyl;
Q is
1) C(O)OH, or
2) (CH₂)mC(O)OH,
where m is 1 or 2;
wherein
A is
3) C₁-C₆ alkyl, or
4) C₁-C₆ alkenyl;
Q is
3) C(O)OH, or
4) (CH₂)mC(O)OH,
where m is 1 or 2.

32. The method of claim 29, wherein the compound is represented by Formula 1C, or a pharmaceutically acceptable salt thereof: 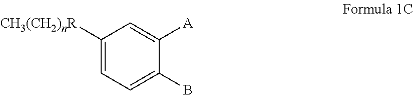
wherein
n is 2, 3, 4, 5 or 6;
R is —C(O)—, —OC(O)—, —CH(OH)—, or O;
A is YCH(C(O)OH)(CH₂)ₚCH₃ when B is H;
B is YCH(C(O)0H)(CH₂)ₚCH₃ when A is H; or A and B are covalently bonded to form a 5-, 6- or 7-membered cycloalkyl substituted with a C(O)OH group;
where
Y is O, S or CH₂, and
p is 1, 2, 3, 4, 5, 6 or 7.

33. The method of claim 29, wherein the pharmaceutically acceptable salt is a base addition salt.

34. The method of claim 33, wherein the base addition salt comprises a metal counterion selected from sodium, potassium, magnesium, calcium and lithium.

35. The method of claim 29, wherein the compound is any one of Compounds I-XVII, XIX-XXVII and XXIX-XXXIV:

| Compound | Structure |
|---|---|
| I | 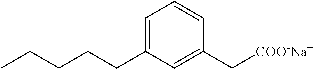 |
| II | 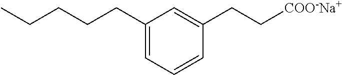 |
| III | 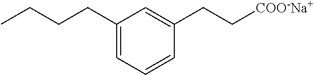 |
| IV | 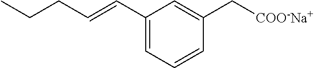 |
| V | 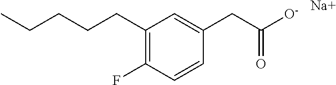 [SMILES: [Na+] ; CCCCCc1cc(CC(=O)[O-])ccc1F] |
| VI | 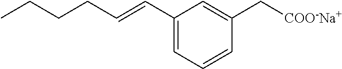 |
| VII | 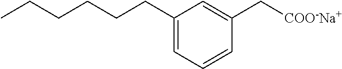 |
| VIII | 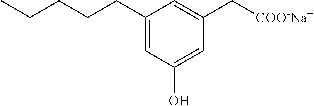 |
| IX | 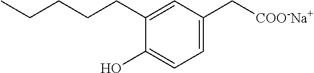 |
| X | 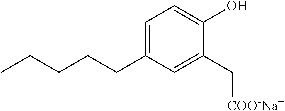 |
| XI | 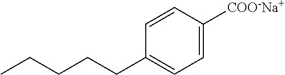 |
| XII | 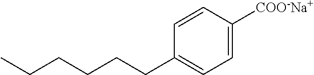 |
| XIII | 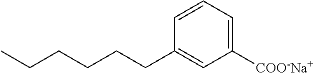 |
| XIV | 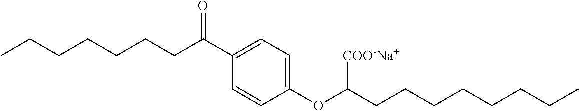 |
| XV | 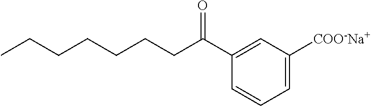 |
| XVI | 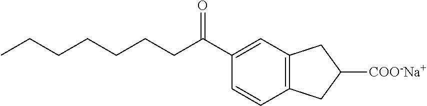 |
| XVII | 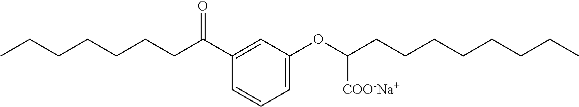 |
| XVIII | 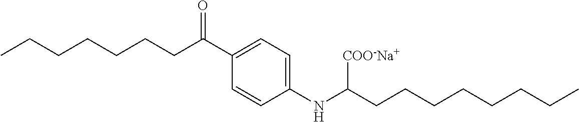 |
| XIX | 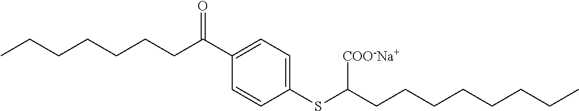 |
| XX | 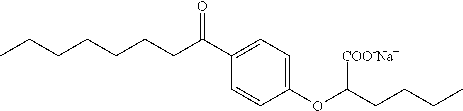 |
| XXI | 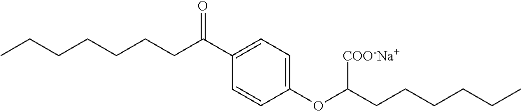 |
| XXII | 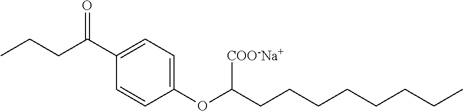 |
| XXIII | 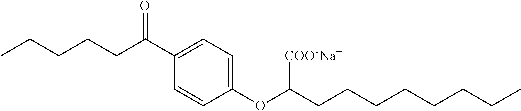 |
| XXIV | 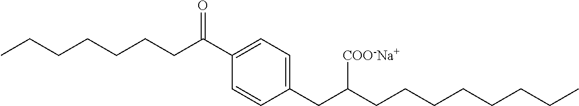 |
| XXV | 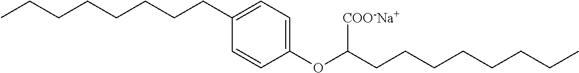 |
| XXVI | 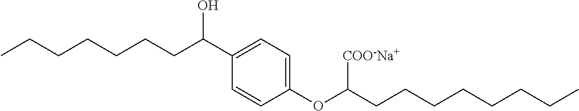 |
| XXVII | 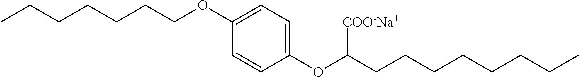 |
| XXVIII | 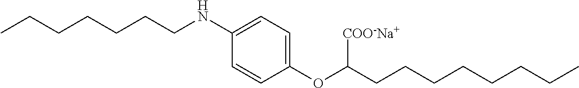 |
| XXIX | 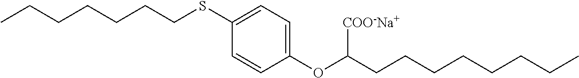 |
| XXX | 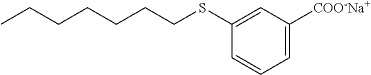 |
| XXXI | 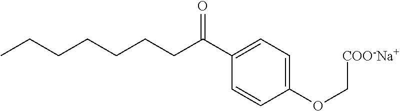 |
| XXXII | 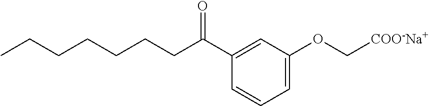 |
| XXXIII | 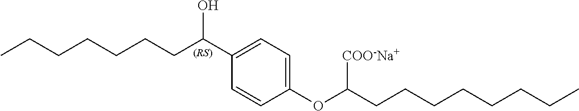 |
| XXXIV | 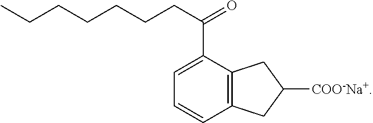 |

36. The method of claim 35, wherein the compound is Compound I, II, V, VIII, XIV, XXIII or XXVI.

37. The method of claim 35, wherein the compound is Compound I.

38. The method of claim 35, wherein the compound is Compound XIV.

39. The method of claim 29, wherein said diabetes or diabetes-related disorder is Type I diabetes, Type II diabetes, maturity-onset diabetes of the young, latent autoimmune diabetes of adults (LADA), gestational diabetes, diabetic nephropathy, proteinuria, ketonuria, obesity, hyperglycemia, glucose intolerance, insulin resistance, hyperinsulinemia, hypercholesterolemia, hypertension, hyperlipoproteinemia, hyperlipidemia, hypertriglyceridemia, dyslipidemia, metabolic syndrome, syndrome X, diabetic neuropathy, diabetic retinopathy, hypoglycemia, cardiovascular disease, atherosclerosis, diabetic kidney disease, ketoacidosis, thrombotic disorders, sexual dysfunction, dermatopathy, edema or a renal disorder.

40. The method of claim 39, wherein the diabetes is Type II diabetes.

41. The method of claim 39, wherein the diabetes-related disorder is diabetic nephropathy.

42. The method of claim 39, wherein the diabetes-related disorder is proteinuria.

43. The method of claim 39, wherein the diabetes-related disorder is ketonuria.

44. The method of claim 29, wherein said use is also for decreasing ketone bodies in the urine of a subject in need thereof.

45. The method of claim 29, wherein said use is also for increasing glomerular filtration rate (GFR) in a subject in need thereof.

46. The method of claim 29, wherein said use is also for increasing insulin secretion or increasing insulin sensitivity in a subject in need thereof.

47. The method of claim 29, wherein said use is also for decreasing insulin resistance in a subject in need thereof.

48. The method of claim 29, wherein said use is also for decreasing hyperglycemia in a subject in need thereof.

49. The method of claim 29, wherein the compound is used in combination with a therapeutic agent for lowering or controlling blood glucose level.

50. The method of claim 49, wherein the therapeutic agent is metformin or thiazolidinedione.

51. A pharmaceutical composition comprising a compound as defined in claim 29 or a pharmaceutically acceptable salt thereof, and a pharmaceutically acceptable carrier.

52. The pharmaceutical composition of claim 51, further comprising a therapeutic agent for lowering or controlling blood glucose level.

53. The pharmaceutical composition of claim 53, wherein the therapeutic agent is metformin or a thiazolidinedione.

54. The pharmaceutical composition of claim 51, wherein the composition is adapted for oral administration.
